SEQUENCE LISTING (128 sequences)

SEQ ID NO: 1 - DNA, 522 bp, Glycine max
  source 1..522: mol_type = genomic DNA
atggcaaaaatgcctttagagcctctaatagtggggagagtcataggagaagttcttgattctttcaccacaagcacaaaaatgactgtgagttacaacaagaagcaagtttacaatggccatgagctcttcccttccactgtcaacaccaagcccaaggttgagattgagggtggtgatatgaggtccttctttacactgatcatgactgaccctgatgttcctggccctagtgacccttatctgagagagcacttgcactggatagtgacagatattccaggcacaacagatgccacatttgggaaagagttggtgagctatgaggtcccaaagcctaatattggaatccataggtttgtgtttgtcctgttcaagcaaaagcgtagacagtgtgttactccacccacttcaagggaccacttcaacacacgcaaattcgcagcagagaacgaccttggcctccctgtggctgctgtctacttcaatgcacagagggaaacggctgcaagaagacgctag

SEQ ID NO: 2 - protein, 173 aa, Glycine max
MAKMPLEPLIVGRVIGEVLDSFTTSTKMTVSYNKKQVYNGHELFPSTVNTKPKVEIEGGDMRSFFTLIMTDPDVPGPSDPYLREHLHWIVTDIPGTTDATFGKELVSYEVPKPNIGIHRFVFVLFKQKRRQCVTPPTSRDHFNTRKFAAENDLGLPVAAVYFNAQRETAARRR

SEQ ID NO: 3 - DNA, 1818 bp, Glycine max
  source 1..1818: mol_type = genomic DNA
agctaaatctaaattatgtattgcaacatattttaaaggaggggcaaaacacactcgatctaccttcaaagaaacttttaagacattataaatatggagcccacactcccttcttcctcagtaagatcatcaacttgtattcctcacttccttagctcctcctcttccttgttcctcctcttacaatggcaaaaatgcctttagagcctctaatagtggggagagtcataggagaagttcttgattctttcaccacaagcacaaaaatgactgtgagttacaacaagaagcaagtttacaatggccatgagctcttcccttccactgtcaacaccaagcccaaggttgagattgagggtggtgatatgaggtccttctttacactggtatatatatttcttttatctcttctcttcttattccttttcttctttaaagaacaagatttttttggggaaaaaaaaagagaaaaaacttaagctgttgccagtgtgtttctttgttattttctgtaatcatggtcacacggtatgtctcttttattggagttttctttacaaatactgaatcattaagcaaatgtccccctttttcggtgcagatcatgactgaccctgatgttcctggccctagtgacccttatctgagagagcacttgcactggtacttaatataaaaattaactaacttaagtagtcatttattacataaataacacaccacacccaccccacatatattgttataccggtttctcttaataacttaacctctttaaaagttatgtactggttatccatctgatattcacgttagatgtctgtgtattttattatcaagtttgaagttttgcaagatatatgcattcctttcatcacatcaaactatggacagccaggaaggtaataacaaatacttacatcacatgaagaaggtacacacatgcctttgtattctgcaaaagtagcttgttccaccataatcccatccaccctaaaatcaaaacaatctaaaagggtgaataaatctcgaagtctcaaacaaacattacagaactacatataaggcaaacaaccagtaaccattcattcattcataggtatctaacagtcaataattttgttgcaaacattctttttgctgcaggatagtgacagatattccaggcacaacagatgccacatttggtaggttcatatgaaaaatgtgtatatatgggaacttactttgatgatcatgagagagatcactgacaaaaaatgatattcctaataatatgtagggaaagagttggtgagctatgaggtcccaaagcctaatattggaatccataggtttgtgtttgtcctgttcaagcaaaagcgtagacagtgtgttactccacccacttcaagggaccacttcaacacacgcaaattcgcagcagagaacgaccttggcctccctgtggctgctgtctacttcaatgcacagagggaaacggctgcaagaagacgctagctatagctaagtagctgctggttttgccactgcaacaaccaaagtagtattgtattgaataaagcgataaaaaggtacaagtacaaggagtttcagtagtggaagttgatcttcacatgtggcttcaaataacttgcagaaagaaggaagataatcattttctagtttgactagtgtgtatgctatgttttactttccatcagatgttgtgtacgttctgtagtataagtgtactacgtgtattattattacctcgtgggtactactatgaggtgatcttatatatatatatataaagaaataagaggtttggtaggc

SEQ ID NO: 4 - DNA, 2000 bp, Glycine max
  source 1..2000: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 5 - DNA, 20 bp, unidentified
  source 1..20: mol_type = genomic DNA; note = Lachnospiraceae bacterium ND2006
aatttctactaagtgtagat

SEQ ID NO: 6 - DNA, 365 bp, Dahlia mosaic virus
  source 1..365: mol_type = genomic DNA
atcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatc

SEQ ID NO: 7 - DNA, 3681 bp, unidentified
  source 1..3681: mol_type = other DNA; note = Lachnospiraceae bacterium ND2006
tccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcac

SEQ ID NO: 8 - DNA, 30 bp, Solanum lycopersicum
  source 1..30: mol_type = genomic DNA
ggatctaagaagagaagaattaaacaagat

SEQ ID NO: 9 - DNA, 1010 bp, Medicago truncatula
  source 1..1010: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacgtactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagagctgccgttggcgtaatataacagccaatcgatttttgctataaaagcaaatcaggtaaactaaacttcttc

SEQ ID NO: 10 - DNA, 3681 bp, unidentified
  source 1..3681: mol_type = other DNA; note = Lachnospiraceae bacterium ND2006
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 11 - DNA, 427 bp, Glycine max
  source 1..427: mol_type = genomic DNA
cgataaaaatgttttaaacgatatatattataaaaaaaaacgtttcaaaaataaatacaaaaatgtttttaaatatatataatttaactcattaaagaaaataaaaatgcaagtgcggtgacaagacaagctaaaagttgcaaaagaaatggcagggctataaggctcacctactcctggatttaccaaattttggttcgtccctatactcgaaaaataaaacaaaataaatttcagtatcttcgtttttgtatgctttgactgtgaggcgaggccaactttcttcttctgtctgagatgaattttgtttgcctcctgtgaaggatgtatcattcaaagtgaatgttttgcaactgccagtagtcccacatcgaccaaatattcttattacagtgtgtttatatagcacctggagaaggaatgggtt

SEQ ID NO: 12 - DNA, 439 bp, Glycine max
  source 1..439: mol_type = genomic DNA
ggcatgtgactttttatttaaattattctcagaaatattaaaatataatcaaatatatttttttataagatactggaaataacatttttaggaatgtaacgaaagccccattacaaacacttgaactcagggatcgatccaacttaattattatctgctacttcaaattcaaatttataggccctacctttatgttttgctctgcactttcttaaatggaaacaagtaacacaatagagtaagatcatttacttgtaagcggaaactgttgcatcaactgcaatattttcgggttagttttataatataacaatgaagagaattgccgagaagatcatatcaagtcccatattccatttattggcctttacaagtcccacatcggtccaaatattagacaaagaaacatttatatatcgtctgacaaacctgtaagatt

SEQ ID NO: 13 - DNA, 23 bp, Glycine max
  source 1..23: mol_type = genomic DNA
tgaccagcggagagagaaagaga

SEQ ID NO: 14 - DNA, 23 bp, Glycine max
  source 1..23: mol_type = genomic DNA
tctcccatcagctcgatcaggtc

SEQ ID NO: 15 - DNA, 23 bp, Glycine max
  source 1..23: mol_type = genomic DNA
gtatactactagtaccatggacg

SEQ ID NO: 16 - DNA, 23 bp, Glycine max
  source 1..23: mol_type = genomic DNA
ttggaagattcctgatgaagagg

SEQ ID NO: 17 - DNA, 23 bp, Glycine max
  source 1..23: mol_type = genomic DNA
ccaatattggaacttggtctgca

SEQ ID NO: 18 - DNA, 23 bp, Glycine max
  source 1..23: mol_type = genomic DNA
agcacgggatgtgcatgtacaca

SEQ ID NO: 19 - DNA, 23 bp, Glycine max
  source 1..23: mol_type = genomic DNA
acagtgaaacgataccaacctgc

SEQ ID NO: 20 - DNA, 23 bp, Glycine max
  source 1..23: mol_type = genomic DNA
ttggtctgaaactgttagttgct

SEQ ID NO: 21 - DNA, 23 bp, Glycine max
  source 1..23: mol_type = genomic DNA
tctcccatcagctcgatcaggtc

SEQ ID NO: 22 - DNA, 23 bp, Glycine max
  source 1..23: mol_type = genomic DNA
gtatactactagtaccatggacg

SEQ ID NO: 23 - DNA, 23 bp, Glycine max
  source 1..23: mol_type = genomic DNA
ttggaagattcctgatgaagagg

SEQ ID NO: 24 - DNA, 23 bp, Glycine max
  source 1..23: mol_type = genomic DNA
tctcccatcagctcgatcaggtc

SEQ ID NO: 25 - DNA, 23 bp, Glycine max
  source 1..23: mol_type = genomic DNA
ttggaagattcctgatgaagagg

SEQ ID NO: 26 - DNA, 23 bp, Glycine max
  source 1..23: mol_type = genomic DNA
gtatactactagtaccatggacg

SEQ ID NO: 27 - DNA, 23 bp, Glycine max
  source 1..23: mol_type = genomic DNA
ttggaagattcctgatgaagagg

SEQ ID NO: 28 - DNA, 23 bp, Glycine max
  source 1..23: mol_type = genomic DNA
tctcccatcagctcgatcaggtc

SEQ ID NO: 29 - DNA, 23 bp, Glycine max
  source 1..23: mol_type = genomic DNA
gtatactactagtaccatggacg

SEQ ID NO: 30 - DNA, 1941 bp, Glycine max
  source 1..1941: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgatacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 31 - DNA, 1568 bp, Glycine max
  source 1..1568: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatctttcctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgcccttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 32 - DNA, 1878 bp, Glycine max
  source 1..1878: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagaaagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 33 - DNA, 1717 bp, Glycine max
  source 1..1717: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 34 - DNA, 1922 bp, Glycine max
  source 1..1922: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccagcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctctcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgtgggtgttcaagaaacgaagcatttcctagaatttttgccttttatgcttttagtttaataataaaacggcagaagcttgacaacttgcaaaggggagggaggacccacacacacaggattacacatgatgctttttttgactgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 35 - DNA, 1842 bp, Glycine max
  source 1..1842: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 36 - DNA, 947 bp, Glycine max
  source 1..947: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 37 - DNA, 1871 bp, Glycine max
  source 1..1871: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgatacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 38 - DNA, 1168 bp, Glycine max
  source 1..1168: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgatacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggaacccctttttctccaacaaaagaatgcaaacagaacttgaataacataaaatttcacatgcactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 39 - DNA, 1388 bp, Glycine max
  source 1..1388: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaaacagaaatttctgcactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgaccgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 40 - DNA, 1311 bp, Glycine max
  source 1..1311: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcccttctttatcaagaaaccattttgccctcttcatctgagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 41 - DNA, 1267 bp, Glycine max
  source 1..1267: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgatcgagctgatgggagagaaaaaactgggaaattttatatttttaaatattttttacaaattttttcagaactaggatgggatagaaataaataaaaaaagtacacaataagagttgctaaaaaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 42 - DNA, 1933 bp, Glycine max
  source 1..1933: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgatacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacactgatgggagagaaaaaactgggaatttttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 43 - DNA, 1064 bp, Glycine max
  source 1..1064: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaattgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 44 - DNA, 1799 bp, Glycine max
  source 1..1799: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgatacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 45 - DNA, 1343 bp, Glycine max
  source 1..1343: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgatacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaattattgtgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 46 - DNA, 779 bp, Glycine max
  source 1..779: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 47 - DNA, 185 bp, Glycine max
  source 1..185: mol_type = genomic DNA
agctaaatctaaattatgtattgcaacatattttaaaggaggggcaaaacacactcgatctaccttcaaagaaacttttaagacattataaatatggagcccacactcccttcttcctcagtaagatcatcaacttgtattcctcacttccttagctcctcctcttccttgttcctcctcttaca

SEQ ID NO: 48 - DNA, 215 bp, Glycine max
  source 1..215: mol_type = genomic DNA
gtatatatatttcttttatctcttctcttcttattccttttcttctttaaagaacaagatttttttggggaaaaaaaaagagaaaaaacttaagctgttgccagtgtgtttctttgttattttctgtaatcatggtcacacggtatgtctcttttattggagttttctttacaaatactgaatcattaagcaaatgtccccctttttcggtgcag

SEQ ID NO: 49 - DNA, 483 bp, Glycine max
  source 1..483: mol_type = genomic DNA
gtacttaatataaaaattaactaacttaagtagtcatttattacataaataacacaccacacccaccccacatatattgttataccggtttctcttaataacttaacctctttaaaagttatgtactggttatccatctgatattcacgttagatgtctgtgtattttattatcaagtttgaagttttgcaagatatatgcattcctttcatcacatcaaactatggacagccaggaaggtaataacaaatacttacatcacatgaagaaggtacacacatgcctttgtattctgcaaaagtagcttgttccaccataatcccatccaccctaaaatcaaaacaatctaaaagggtgaataaatctcgaagtctcaaacaaacattacagaactacatataaggcaaacaaccagtaaccattcattcattcataggtatctaacagtcaataattttgttgcaaacattctttttgctgcag

SEQ ID NO: 50 - DNA, 95 bp, Glycine max
  source 1..95: mol_type = genomic DNA
gtaggttcatatgaaaaatgtgtatatatgggaacttactttgatgatcatgagagagatcactgacaaaaaatgatattcctaataatatgtag

SEQ ID NO: 51 - DNA, 318 bp, Glycine max
  source 1..318: mol_type = genomic DNA
ctatagctaagtagctgctggttttgccactgcaacaaccaaagtagtattgtattgaataaagcgataaaaaggtacaagtacaaggagtttcagtagtggaagttgatcttcacatgtggcttcaaataacttgcagaaagaaggaagataatcattttctagtttgactagtgtgtatgctatgttttactttccatcagatgttgtgtacgttctgtagtataagtgtactacgtgtattattattacctcgtgggtactactatgaggtgatcttatatatatatatataaagaaataagaggtttggtaggc

SEQ ID NO: 52 - protein, 174 aa, Medicago truncatula
MARMSQEPLIVGRVIGEVLDSFTTSMKMTVSYNKKQVFNGHEFFPSTINTKPKVEIDGGDMRSFYTLVMTDPDVPGPSDPYLREHLHWIVTDIPGTTDATFGKEVVSYEIPKPNIGIHRFVFVLFKQKNRESVTASPSSRDYFNTRNFASQNDLGLPVAAVYFNAQRETAARRR

SEQ ID NO: 53 - protein, 171 aa, Cajanus cajan
MARMPIEPLIVGRVIGEVLDSFTTSIKMTVSYNKKQVYNGHELFPSTVNTIPKVEIDGGDMRSFFTLIMTDPDVPGPSDPYLREHLHWIVTDIPGTTDATFELVSYEIPKPNIGIHRFVFVLFKQKRRQCVTPPASRDHFNTRNFAAQNDLGLPVAAVYFNAQRETAARRR

SEQ ID NO: 54 - protein, 174 aa, Pisum sativum
MARMAQEPLIVGRVIGEVLDSFTTSMKMTVSYNKKQVFNGHEFFPSTINTKPKVEIDGADMRSFYTLVMTDPDVPGPSDPYLREHLHWIVTDIPGTTDATFGKEIVSYEIPKPNIGIHRFVFVLFKQRARDSVRATPSSRDHFNTRSFASQNDLGLPVAAVYFNAQRETAARRR

SEQ ID NO: 55 - protein, 177 aa, Arabidopsis thaliana
MENMGTRVIEPLIIGRVVGDVLDFFTPTTKMNVSYNKKQVSNGHELFPSSVSSKPRVEIHGGDLRSFFTLVMIDPDVPGPSDPFLKEHLHWIVTNIPGTTDATFGKEVVSYELPRPSIGIHRFVFVLFRQKQRRVIFPNIPSRDHFNTRKFAVEYDLGLPVAAVFFNAQRETAARKR

SEQ ID NO: 56 - protein, 179 aa, Brassica napus
MENMGSRVIEPLIVGRVVGEVLDFFTQTIEMNVSYNKKQVCNGHELFPSFVSSKPRVEIHGGDLRSFFTLVMIDPDVPGPSDPFLKEHLHWIVTNIPGTTDATFGKEVVSYEFPRPNIGIHRFVFVLFKQKQRHVIDISPNIPSRDKFNTRKFAIEHDLGLPVAAVFFNAQRETAARRR

SEQ ID NO: 57 - protein, 172 aa, Gossypium hirsutum
MAREVEPLMVGRVIGDVMDSFIPSIKMSVTFNNKQVFNGHEFYPSTVVTKPRVEVVGGDMRTFFTLVMTDPDVPGPSDPYLREHLHWIVTDIPGTTDATFGREVVSYENPKPNIGIHRFVFVLFKQKRRQIIKSPCSRDNFNTRRFASENDLGLPVAAVYFNAQRETAARRR

SEQ ID NO: 58 - protein, 172 aa, Capsicum annuum
MARSLEPLIVGRVIGDVLDSFNPTIKMTITFNNKLVCNGHELYPSAISARPKVEVHGGDLRTFFTLVMTDPDVPGPSDPYLREHLHWIVTDIPGTTDATFGRELVSYEIPRPNIGIHRFVFVLFKQKRRSSVSQPTARDHFNTRDFAQENELEQPVAAVFFNAQRETAARRR

SEQ ID NO: 59 - protein, 172 aa, Nicotiana tabacum
MARSLEPLIVGRVVGDVLDSFSPIMKMTISYNNKLVCNGHELLPSVVTARPKVEVQGGDLRTFFTLVMTDPDVPGPSDPYLREHLHWIVTDIPGTTDATFGRELVSYEIPRPNIGIHRFVFVLFKQKRRQSVSSPTSRDHFNTRNFAEENDLGQPVAAVFFNAQRETAARRR

SEQ ID NO: 60 - protein, 172 aa, Solanum tuberosum
MARSLEPLIVGRVIGDVIDSFNPTIKMSITYNNKLVCNGHELFPSAVSSRPKVEVQGGDMRTFFTLVMTDPDVPGPSDPYLREHLHWIITDIPGTTDATFGRELVSYEIPRPNIGIHRFVFVLFKQKSRSSVSQPTSRDHFNTRNFAQENDLEQPVAAVFFNAQRETAARRR

SEQ ID NO: 61 - protein, 172 aa, Solanum lycopersicum
MARSLEPLIVGRVIGDVIDSFNPTIKMSITYNNKLVCNGHELFPSVVSSRPKVEVQGGDLRTFFTLVMTDPDVPGPSDPYMREHLHWIITDIPGTTDATFGRELVSYETPRPNIGIHRFVFVLFKQKSRSSVSQPTSRDHFNTRNFAQENNLEQPVTAVFFNAQRETAARRR

SEQ ID NO: 62 - protein, 173 aa, Zea mays
MSRVLEPLIVGKVIGEVLDHFNPTVKMVVTYNSNKQVFNGHEFFPSAVAAKPRVEVQGGDLRSFFTLVMTDPDVPGPSDPYLREHLHWIVTDIPGTTDASFGKEVVSYEIPKPNIGIHRFIFVLFRQKSRQAVNPPSSKDRFSTRQFAEENDLGLPVAAVYFNAQRETAARRR

SEQ ID NO: 63 - protein, 173 aa, Oryza sativa
MSRVLEPLVVGKVIGEVIDNFNPTVKMTATYSSNKQVFNGHELFPSAVVSKPRVEVQGGDLRSFFTLVMTDPDVPGPSDPYLREHLHWIVTDIPGTTDASFGREVVSYESPKPNIGIHRFVLVLFKQKRRQAVTPPSSRDYFSTRRFAADNDLGLPVAAVYFNAQRETAARRR

SEQ ID NO: 64 - protein, 173 aa, Hordeum vulgare
MSRSVEPLIVGRVIGEVLDTFNPCVKMVTTYNSNKLVFNGHELYPSAVVSKPRVEVQGDGLRSLFTLVMTDPDVPGPSDPYLREHLHWIVTDIPGTTDASFGREVISYESPKPNIGIHRFIFVLFKQKRRQTVTVPSFRDQFNTRQFAEENDLGLPVAAVYFNCQRETAARRR

SEQ ID NO: 65 - protein, 173 aa, Triticum aestivum
MSRSVEPLIVGRVIGEVLDTFNPCVKMVTTYNSNKLVFNGHELYPSAVVSKPRVEVQGGDLRSMFTLVMTDPDVPGPSDPYLREHLHWIVTDIPGTTDASFGREVISYESPKPNIGIHRFIFVLFKQKRRQTVTVPSFRDHFNTRQFAAENDLGLPVAAVYFNCQRETAARRR

SEQ ID NO: 66 - protein, 173 aa, Sorghum bicolor
MSRVLEPLIVGKVIGEVLDHFNPTVKMVVTYNSNKQVFNGHEFFPSAVTAKPRVEVQGGDLRSFFTLVMTDPDVPGPSDPYLREHLHWIVTDIPGTTDASFGREVVSYETPKPNIGIHRFIFVLFRQKRRQAVNPPSSKDRFSTRQFAEDNDLGLPVAAVYFNAQRETAARRR

SEQ ID NO: 67 - DNA, 2000 bp, Zea mays
  source 1..2000: mol_type = genomic DNA
aggtgttttcaataaagatatatgagataatgtcaatttgtctggagctgaatcggttgggagttaaatgttagtttgtttggcccctagattttcacttaatttccttgtgactttatgagaaggcaagtgaatgaacctgcagaatcaacttcacccttaacattagtagcaagttagcaaatcgtaggacgtgcatgtatttggggatgattctactatttgtggaaacaagagcactaacctttgcctaatgaatagctcataattgaggatctccttttccctcaaaagaagtgttacgagcagagcactcttggaaacaggacctctttgtgtagtggagggtgccctgatccacttgagagcttaggtggtgtttggtttctagggactaatctttagtcccttcattttattccattttagttcttatattgtcaaatacggaaactaaaatagagtcttagtttccatatttagcattttaagtactaaaatagaataaaatgtagggactaaacattagtccctagaaaccaaacacccccttagtttatgccctcctcttgcacttgcgtgacaggaataatcaccagaagtagcaagtccgctcaagtgcaatgctgtttagggcatgttcttttgcaccccatagctcagcagagtggtgcttcagcgtgcctcgcttttttacctcaagagaaaaagatgctagctcctgggtgcagttgtgctccagctttgagaatacccaagctaagctcactctccagctgcaaaaagaaaaactaaaatatgtgttgaaacgcaaaataaaatacatgtaaaaataaagggataatcaatcaatggagagagaattcacattaacgttgtatgcttctttatataaagggacaggcaaagaatacaagctaaagaactagcataatcaagctgccatctttgacaccgttcctacattcactaccacacattatgtctctaagaatttctgaagaaaatgaaataattaatgcacgtttagtgaagattctctatggtacactactaataatgtcttcaattaactcctcagtgctaaatatatacaaattcatactatcatttttgtacacaaaacataaattgtgatgtgatttgtttcagtaaatcacaatcaatacgtcgattagatacaaaaattgtcacctcacttctggcttctgcgctcacatagcatgaacaaaaaaaaaacctggcacactaataataatcaaacctaagactagatcatcacccatgttccagagaaaaatactgtgaccgcctctatagggaataaaataatccatgtgggaagccagtgcaaaaccgaatgtactaggcgaaaaaaaaatactaaaccatttctgcattaccaagtcaaccaacaagtagctgcagctccgaacggaagtcaagattttacatgcatcccttataaaccacgaagctaactcccaaaacacctgagaaatgctacgatcttttatatttcaatgtgctcgcttggcttgatcgatgtctgaaggaagtacgttatcagtggatctctgtcgtcgttcaccggagaaactttaatgatatggatgtaacaaagagctttttcctattttgccaaatgtggattctaggcacttaactatttcggcctgaacgcccatgcatatataataatataaatattgtgaagggaataaatatctttgagatagatgtggaaattggaatatatcggataataaggaacttgtatatctgctgcaagtgcccctattctatatattgtgaagaaacaatctgacatgttaagctatatatgatagtagccaccgtacgagccgatagagacgctcaaacctgaactttttaagagctgtaatatatgatgatgatcggtccatcacactcagctcgatcgatcggaatatatagctggccttgacgtctataaatagaggccaaacgccctggtc

SEQ ID NO: 68 - DNA, 2000 bp, Sorghum bicolor
  source 1..2000: mol_type = genomic DNA
caacttcacccttgatactatactaatagcaagttagcatgcaatgcaagtatattgctacagagattagagtatgcaaattataggacatgcatgtacttggagatggttctgctatttctggaaacaagagcactaacctttgcctaatgaagagctcataattgaggatctccttttccctcaaaaggagtgttatgagcagagcactttgacacagggccactttgtggagtagtggagggaggcctagcccacttgagagtttagtttatgccctcctcttgcacccgcctgacaggaataatcaccaggagtggcaagttcgcgcaagtgcaatgctgtttagggcatgttcttttgcagcccatagctcagcagactggtgattcagcgtgcctcgcttttttacctcaagagaaaaagatgctcgtgggtgcagttgtgctgcagctttgagaatacccaagctcactctccagctgcaaaaagcaaaaataaaatatgggtttcaacgcaaaataaaatacatgtaaataaagggataatcaatggagagaattcacattaacgttgtatgattctttataaaaggatgggcaaagaatacaaccgaaagaactagcataatcaagctgccatctttgacaccattcctacactaactaccacacattatgtctctaagattttctgaagaaaataaaataaaatgcacgtttagtgaagattctttggtacactactaagaatgtcttcaattaactccacaatgcaagtttaaatacataaaaattcatacttattatcatttttgtaaacataaagtgtaatgtgatttgctgtttggtgcaatgtcatgggcagccaataggctgccaaggccgacgggctgatagttcggcccagttatctttataagtttaattatctttgtatttgcaatttatattagccccagataggttatttagcaaaagaggtgaagtagaagggacataaatatctgtagactctattgatataatcaagcaatgaattatgaagaaacaattctagtctccttcaatacccatccctataacatgcaaggtacacacatagacatagctaaatctccaaactggtttcacacatcattagtagggatgaaaacggtacggatatttttcgaccatattcgagaccaaattcgtttatagaggtttagatctgtccgtatccgagtccgaatatttaacatccgataccgtattcgtatccgaatatttaaatcgtatatttatgatgtcgatatccaatcatatcttatccgacatgattaacattatccgtattcgaatccgaatccgaccagaaatataaaaacaaatatgatatcggtgatatccgtttgtatccgatccgttttcatccctactcattagagaagtatagcatgtatatgggcataggtctcatatatatatatatatatatatatatatatatttcatagtgctcgcttgatctctaaaggatgtgatcagttgatctgtgttcaccagagaaactataataatatggatgtaacaaagagccttttcctattttgccaaatgtggattctaggcacttaattatttcggcctgaacgcccataaatcttctgaagagaataaatatctttgagatagatgtgaaaattggaatatattggataataaggaacttgtgtgccgcatgcaagtgcccctattctataatccagggtccacatcacacctggatgaaaatgactgggactatatagcaatcttaacataacatgttaatctatgataatagccaccgtacaagacgatagagacgctccaacctgaactttttaggaacttctgtttgaaaggtgtaatatatgatgatgatccatcccactcagctcggaatatatagctggccaaacagatagataaggctcgtgtcttgacgtctataaatagaggccaatgccctggcct

SEQ ID NO: 69 - DNA, 2000 bp, Oryza sativa
  source 1..2000: mol_type = genomic DNA
gcatcagattaagacagcaacgcagaattgcagacagagagaatgcatggaggcagctagcaagcagcccctgcctgttggttctctacacggtttcaatcaatcaccagctaattggcagacattcaaacaaatcgcaacaccgtagtatagtacttttaattcgtcttcgagatacgactttaatcattaattgccttacctcaactttatgttcggattacatatcatgtaatattataaaaaaatctaatggtgtaataaatgttataaaataaacttagatgcatagttccggatatatgcccctccggtttttaatgtacaacacttaaattttggatgtgtgttcgttttgaatcaaatctgaaatagtatgttacttttaaagtgcctttaaagataaaacaaatcacaataaaaatacatatatttttaaataagacagatgatcaagcatatattcaaagtcaacagatcagtcatacattattagaagagaatttcttgtatgcccttgaaatttaagtcaatcttttctatgcccttgaattttactcactctcttgtatagttgtataccttttaattatgatttcatctcttttacatcttttccgttagttgaccggttccctttaaaaattagaagtttgtgtacgagactattatatttctgagtttgttgtgcgatgtactatttacaaaaagaaattagatgtgcaagaaaaataatatttatttaaaacaatatgtgtagcatatttttgtgatgtaacatatttgccacctattgtatatattatctaaaagaaatcctatacaagaccaattgttttttttcaattataaaaaagccgcacaagctttttcatgaatatattaaattttctaagtacaatacttcctcatagaaaaaaacttataatattatagtcccacaataaattactatatttccaacgttagcatccacaaaatttctaggcatgtacaattgtaaaatgacctttttacaggcattaaacagttaactaacggtacggaaatggtataaaacgggttaaaattaaaattcagaggtatataaaaaatgagcgattagctaaacttttaggagtataaaagaaattctcttactgaaaaccagagggagtactacatataacctcatttttttacgggttgataagttgcggaaaaaccaacaaattagcaacaaatatgagtaaaacttgtatacatgtgtcttcttagcgatttaaaaatcaatgctgaaaataaattacaataaaattaaaaatctcaagataatctctaaaatgtagttttaaaatttaaattttgattgcgactgataagaaaaaaaaacaaatgatgggaggctatatcaactgtcaaactggctaatttagaaagacgacatcgaattcctacagattggaggcagcaaaagagagcccgtctctgccgggtgcgtgcatgatgctcgatcatatcccatctctcctcacttcatcatcaacaagaacaagaggaactatatagctgcaagatctagactgaaactactatagcaactaactgtactgtagctagattacgcttagctatagctgctgctgcatctatctgtaaattccaactacgacgtcgactgctgcaagctagcttggccggctagctagatagctagctagctcaaaagagaaagctttgcgaaataggagtaactagctagctctagctagggcgccatcgatgcgtgaccaccgagctcgcctcgtccacacgtacaggaagacgatgcagaaagcggccggcatgcagtacactgaccgagctaagagagagagagagatgatattattcttctgcagctaaattaaagtgaaagttggacatggacatggacatagtaattttgcatggccatcatcttgccctcctatataaagcggccatctcactctcaaccacagctcgatccagcagccctg

SEQ ID NO: 70 - DNA, 2000 bp, Triticum aestivum
  source 1..2000: mol_type = genomic DNA
cgcgtccaaggcacatgcgtctaaatcaacacaacaagaaccgcggtgaagaggaaaaaaccgggtcttcatacatctcaatatacgcttgagatggacatctatgaacatggctaacaaagtctacaacagccagaggggcaactaaggttgctccgcaggagatacattttcaagcaagaaaaaacataactggtcagctaaaataaaatcggaaggaaactatttttttaaaagcgtcgtcaaaaagggtaacccatcatttgatcccctgctgagttgcccagctcggcagccatcaaatcagaatgctgccttatccttcctaggttggcacgctttgtttaagcttagtggaatccaaaagtgaaaatggtacggcgcagagaattcacgcagaacaatgcttaaataactgaaaaagaatggacggaaagagcaatgacaattagcataattaagctggcggacgggcagccatctttgacctgtcttacgattttacccacgcgaaatgagatgcacatgggaatgaactgttttcgtttagctagaccctccgcatcaatcacaccactatttggtctgtgtaccgtgtgcgacaacaattggcagtaaacgtacaaagctggtccagtgcgcagtatccagacatacataaaagtacttaccgcaaaatgccagacatggaaattgacaatcagataataatttcaaaaaactgctccctccgttcacaaatgttcacaaatataagatgctttctatattgcaatatgaactgcatacagactgaaatgagtgaacaaacacattaaaacgtagctatatacatctgatcacaaaaaagttactccctcagtaaactaatgtaagatgttttgaactgcaaaaacatcttatattagtgtacagaggtaatacaacatcttatatttgtgaacggagcgagtacataagatcagaaattatccaaataacagttacaaacaccaagcccaacgaggccgttccagtgggtgtcattgtcactaggattaaggaggaaacctcgctttggagtattgcgggggcgaagcacttgagtaatgtaatgccgcgagagtgattattgtaattcggccatgagccttaaacctctaaaacttctctcttaatcaatgaaaatggcaaatcttttgccttgtttcaaaaaaaaacaagagcgaataggagcaaactagagacaagtagagagggacgccagtggagcgcacgcgctctgtttttgcacccctttgggctttggtgttctccctgccgcaatcacgtttgagcgcagagttggagcaccatatctccctgctctgcactcactactgcgctttctcataatataattgcaataccagtacagatcaacaaataattttccaaatacatattctttttactggatagaattaaactatatgcatgcatgcattttcaagagagaagaaaaaagaaaaaagaagcatttcgacagaaatataaatacatagtacgtattagacttaatggtataggccatactcaatcatcactaagacaattttgaaaaaggcctttaagcaattgtcaatgcgctcaaacagtgtggcttctaataatgtcctccggaaggatcgatgaactgtttcaggaacttgtactatggtacaattgatctacctcagcttgggactttgtaggcgcatacatggcaatcgggctttcttcttttaccaaatgtgtggattctgtgcattatgaacaaactttttccaaaacccacctgtcacgaatatacagacttatgctagttttcaggtttgaaacctttttaagatgtatgctccagttttatagtgcgcaaggacggaataatcaaacatcacctagtgataatctagctttaaaacttcaccctcctcgcacacagctcactcaccccgcggctctccacggctataaatagagcctctacgccacccaacctc

SEQ ID NO: 71 - DNA, 2000 bp, Hordeum vulgare
  source 1..2000: mol_type = genomic DNA
tgcttttgactggatagaacatatatacatgcatgcattttccagctcccactcttcctcccctcccttcgccgtcgccggcgtggtccgtcgagccctgcccgtgcggcgctggcagcggcgggccgacttttccccatgcgtgtgtcgggaggcacgggggccctctgacgacggcggtgctgctcggccgcggcaccgtggctgagcggcggagatcctgtgggccacgggcacccttgacgcggtggcgcagtcgttggtcgcggggcggcggcgggcggacttttccccacgagtgtgtcgagggccgcgggggcctctaacggcggcggtgctgctcggccgcgccggtgtggctcagcggaggagatcctgcgggtcatgggcgcccttaatgcggcggtgcagccctttgtcgcggcgcggtgcggtgtgcggctggccggcggcgccagctcggcccagatctgtgcccttttgggccccatctaggctggggcaggccggcggttcaggtcgcggcgatgcgactgcctggtggtgcggggaggcggttgtggctgcggtggtgatggctctggcggccggcttgctacagcgtggctgctgggactgtttggacccgttgggggtctgatcgggcctgtgatggcctggcaattccctaccgctacatccgttcggctgcgcgcggaggtgaaggttgtcccctccgaccgacggtgttacggttcccccgagcccaatatctcctcgtcctccctccaggtcttgtgttggtggcctcagtgagacggtgaagttggtgggatgaacgtggtggcataggttggtgggtggcatggtgggaggtgcacttccgattgtacgtggtgacgatggtggtttcggggcgggagaaatccctaacggctcgtccggcaccgacacggttgtgctcgtgggcgccgccgaaccttctagaggggcgttggtgataccccttccccactgccatccgcataccgggggaaaccctacgacttgtccgagtagcagcgaagtcgtcatcgctttccttcctgaaggtgttgcttggtacgcgaggcttcggagtgcttggcatgcggtggtacttctcgaaagggtgcggcggtagtcggccatcttcgcttcgttgagctgccggagtcgacatttttttctcttctctttctttattctttttccttgggcttgtttgtgttgcggcccccagcgagttggttgttgctttataatataaagagaaggaaaaccctttttcgtcttagagaaaaagaaaaagaaaaaaacattttgacagaaatataaatacatactattagagttaatcgtgtagaccatactcattatcaacgccaagggaaaaaaagagtagaagacttttaagcaattttcaatgtgctcaaatagtgtggcttataataatgtcctcccgaaggattgatgaactgattggggaacttgtacaatggtactccctctatttctaaatataagacgtttttgcagttcaaatatttagaaaatagaggatagtacaattgatctacctcagctggggactttgtaggcgcatacatggcaatagggcttttttcttttaccaaatgtgtggattctgtgcattagcaactcagaataagcttctcctgaaacccacctatgtcgaatatacaggcttatgttagttttcaggttcgaaacctttctaaggtcacgcccccgttttgtagtgcgccaaggatggaataatcaaacatcacctagtgataatctagctttaaaccttcaccctactcacgcacagctcactcagctcacggctcttcacagctataaatagagcctctacgccacccaacctcagcaccaaccatcaggctatactacctcattcctctcgtgcagctactagctacctgcagcctgtaggtacttgtcatcattcatcacaaacttttcagttcaagcta

SEQ ID NO: 72 - DNA, 2000 bp, Solanum lycopersicum
  source 1..2000: mol_type = genomic DNA
ttagaaattagttattagcatcttaacttgcacatgataggtagttagatataattagtcacattataaatatgctgtaacaaaccaatattgtaattcaattttctgcaatatacaatacacagttttctcaatgattttttcttcttcttcatcttctccatcttctattctcttcatcttcatagatttagttacagattttcaataattcaacaaccatcaccatactcacaattactaccacctccaccatcactatcaaccactacaatccttgcgatcaatctctactacaaaccaatgaaccattttcattatcataactagcacagctactatcatcaacacatcatcaattaccatatatattcttcacccatcgctgttaatatcactaactattaatatcaatcaacttcaccaggacaatcaccatcaccactattaaatgtcatcatcacaccagtcattacaaaactaacagtctccaacattaccagtaatcactaacaacaaccattattacaaacaatctctacttatttacttttattcaaatatttatttagacaaaactgattttagtaaaacaaatgagatcaatctttttctcgtgattaatttttaagttggaattagttccaaaatacatttaatatagacaaatatgatactcccaccgtcccattttatgtaaaaaaacacgtctcattttcttatatggtaagtatttaaaggtataatttctcttttttacctttattgatcttaattttctaatacatttatgagaagagagaaaaatgagttacttttttaaagaacgatttgataaatattttaaaatcttcattatttcttaaattttgtgccagatcaaatgttgtcacataaaatgagacggaaaaagtaaaacatatcaaacacacccttaatttaaaatagtgtaggtactacctaaaagtggaagttaattatttgtttccccaaaattaaattttacccttggacagcaattcctgtttaagggttaattgtatagggacatacattttcttctaatagtccagggtagtttggttttcgatatgtggaaaattatctcgacataaaatgctacagtaactaattagtacaatatttagtttgtatttaccattacatttagctccacattcacataaattggtagtacaacatttaatcttctaatttgtactataacattttcattaataaaaagtatggtttctatcttcaccattagcgtaacgttcgagtagagatatacatatttattattataatatacgattgcaaatgccaaagatggctaattttgttttgagagactactgcattaagtaaattttttcagagacatgtataagattaagtctattgccaattctcaaatattactcttttttacttattgtggttatttatacatattaagtgaactttcttttaagacaaaaatgtgaaagaaatgaatttcaaatttgattcaattccataaaatagctcaaatcggaggaggaattaatattcaagtcttataaggaaattattcatcgatcatgattatttttccatgttaaattgattaaatcttttttcattcttcaacatatctaatcttctaccctacaacaagctctcacctttcatagtatttatatagactatatattcgtataaaatatttttcttcatatcgaacacacatgatctttttaggatagagggagtattttttaaaaaaaaaataatggggcaaacgcaaataaaatagaacacatatatattctttctctagctgctaattaagctatgactttataattttgtagcacgagaagagaataacctttttgtgcttttcatttctttaatttggttccccattttttgaactatcaatattttagtccctatcccatctgactctctaatgatcttagggccactataaatattggtattttgctctt

SEQ ID NO: 73 - DNA, 2000 bp, Gossypium hirsutum
  source 1..2000: mol_type = genomic DNA
aaatatttttttgggtcccttctagccttggacattggtggccgcactcccaaactacccctaaggttgagcctgggtaaatatcttatagggtatgacatgagaaatcaccatgtatcttatttattttgtttttaaaaaatatcaattaagaatatttaacagtttcttaagtccacttgtaaaattaaaaaaaaactctactattaatatatcaaataataactcataataaaatatttaaaaatatatatataaaaaaacaagtaaaaatttaaacttaaaaaaaatacactacaaatatactaaatattaatcattatattgtttatgaattcaatctactttttaatctctctaggattagccatttctttagattggagagaggtaaaaaaaaggaataacatggtccagttccttcatgtcctatcctatgcccatttaataagatagagagggaaagatccggtataggtatctctagaattgttgttggtacaatttttaagtcaaaagttgcttacttatgctttatttatttgattgttttgaaaaggattgcttacatttattattattattgaggaagaagattgcttaccaatgttaaacgggtaaaaaattttggcaacattaaaatagagttaatttagaaaagtgacattattgttctcatccagtccatcattattgttttatgttgtaattcggacactttttttttgttgctcaactgtcatattactttttggtcgcttaattttaaaaagttacaaaatgattattaaattatttgaaagttttcatttaatttattggactattaaaattattgttgtttaaccttctttgttcacattacgtgcaccgattaaaaactttccttcttctcttctacaatttaaattattttcatgaattaactttgaacgtcatgaatttacgaactaaaatctaaacaattttcttctccgatagttgaccttgattataacttgaatctaaagtatgttcttctactcttcgatgtgtattgatccaccataccaatcgtcaaatagttgcttggagctcactgtccaaacttaaaaaaaaaacactcaatagtttagtgaccggaataaaaatttttaaatagtagagtgacttaaataaaactttcaaatagtccagtggccattttgtaactttttgaagttgaatgatcaaaacgtaaacttactaatagtttggtgaccttgggtgtagttcacccatttttaaataacatttttttttctcaattctttttaaacttaaagtttttccttgtctcatgtactaaacgtgtccctaagttgatgctatagtatgggagacccaaaaagcatatctaacgagttcccattactagttttccttttttggggggggggggggcctgctgaatctcccaaacatgaattacaattgttcaaatttaattctatacttgcatatcatatcttgaaagtgattgtttgaaatcagtggacttaaaaacctgatttagaaactctcttgtttttgttaacagaaccacctgttttttgtgttctaataacttccaccagtgcctgtcataaatcggtatatagtctttagtactagaaaatttggtgtggctagaaaaaagaataaggtttatctctatagaattatttgatcgggtgcttcatttcaactttaacatcagtgtcagaatatatagatataacgtatattatttgcattgaatatgtttaaaaatgagttatggcatttaagaataagtttttatagcaaaattatattgcaaatggctcacctttgagcaatttagtataggttctatcttaatggtatagagttgagtgtctagcttactagatatgaaaagaacaataaaacctttagtttttagtgtttttttatgttccattttcttaacctcacaacctaactaatggatctcattaagttagcttgtgggtggtgat

SEQ ID NO: 74 - DNA, 2000 bp, Capsicum annuum
  source 1..2000: mol_type = genomic DNA
gtgtagctccgcccctgggatatttcattaatatggattatgataagcttattaagtagccgtttggccatagaaattattttctttttttcgaaaaattatttcactttagtgaaaaaagtgaaaacacgttgcgtttgaccatgaatattccaaatccaattccaaaaaattatttggaaaagtgaaaacaagttttacttgttttcacttttttcactcgtacttctttcacaaaatttcaaaaacaactttaatttatattcatggtcaaacacaactccaactccaacttcatcttcaattctaactccaactccaaaaaaaaaataattttcatggcaaaacggggcctaagattcattaactcttaattagagaccttagattcgaattctacaaaaaaaaaatcttgttagataaaaaatgaataacgtaatgtgtgaattcaaatttaatgtagaaaaaagaagaagatatcaaacggaaagtaaaagaatagttcttggatatgtcactgattttttacttccaaaaacgttttttaaacttagagaagtaagtttactaggtacttacaattgtcaaagttgtaagttatttggtacatgtgccccccttaagttagaagagaaagtacacaataagttggcataaaaagaaagaaaaaagactcaatgatgagatggttcaaagacaaagttcaatatggtacattattggtcaatttcatttgatcatgtgatagttttttgattggatagtgcctttaattagaaatacttacataaaattaaaaaggggtgtggatcgtcttcttaaaaatttatcagtctagaaaactgtcaggttttctctttgattcgttctcatcgatcggcccccctctgtcatggagcgccacataaaaccccttagaactcagcttcctcactaaaattttgtgtgatactatgagagtatggctctgatactatatagatatatatatcataattcaaatctcgactgcgtctgtattgacctaatagagtgcacatatttattttttggattataatatcattgagttcaaagaaatgcacatacagtgtggattagtgttacatcttatacaacattttattcttctctttttaatatgagatttgtctaagatatgatacacatcattctttaaaagcttaccagcctaactaaacgtctatttttttttttgccccccgccccccaaatctagattgcgtctgttttgacctaacagagctcacatatttgtttgttggattacaatgtcatcgagttcaaaaatatggacatactgtgtggatttgtattacgtcttctataacactttattcttctctttttaatatgagattcgtctaagatattatatgcatcattctttagaaatttaccggcctagctaaacttctactagtcttttgaccccctccccccatcaacttgctctccgttatgagcgtcacataaaatccctccccccaagccccgccctcttcaacttgctgtccgtcatgagcgtcacataaaattacaccccctcccccccccaaaaaaaaaagttgtagaataggtgtttggtggggtggtgtgtgtgtaggatgggttgagtaggaccaagatacaggggcggagccaggccttgttagggggttcggccgaactcccttcgacggaaaaatacaccatttatacatggttaaaattatttttatgtatttatagtaggtgtcgaaccttcttcggctaggttttcttcgaatattgaacccccttaattgaaatcttgatttcgcctcttccaagataaaaagaattcttgtaataactttttgaagtttcctaaggtcccatatttttataccttattttaaaaatttattctcctacactatgatggctaacttaatacacacacatatataaaggtgtccctctaatctatttgtccaatacaaaccattcttctaatt

SEQ ID NO: 75 - DNA, 2000 bp, Brassica napus
  source 1..2000: mol_type = genomic DNA
agatgatacactggatttatagatattataaattaaataaatataatttaatgttgtaatattatacctcttatttaaatatttgtcgatgttaacttttaaaattataaagattttttttttaaatagcaaaaatcatattatctaacaatgattaatctttactaccttaaactaatgaaaacaaattttaaaatgtatagtttattataaaaattaaacaaaaactaaatgtttaattatttactcgataatataaatctatgaagcgaaaagtttaatttttaaaaaactttctaaatttgtaaaatgttacaatatttttgaatatgacaataaaacaatattttattaatctttatatatatagttacgattttaataataaaataataatccgaaaatatatatatagaagaagatacagatacatgtgaaagtttgaaacaatctattcaatgaaaaaatataccgtaaacttattatgttttaaaaattgataaacacatatatattataatatataccaatttagaattgaaaaaaaaaatgtttatataaaaataaatgaaagcaaaactctgtgcggttgcgcggatcgagatctagttattgttaaaacaaaacacttgggtaaaattactaaatatgaaaaagttaagatgttattaataattaatgataaagtggacatagaatatacgtagaatatttcttttacaagaaagaaatgaagcaaatcattgcatgagatgtacttcattttttgctttattttgaataaagtgcatttatgaaggtgaaaatgaagtcaaccattaaaaatgccctattagaaaaaaagaataggacccgcacgtcaataatttgacaaaaaattcaacacaactggtacaaaatcgaaagaacaaaaaaatcaccataatttttttcattcgatctggtcaccacttagatatataaactcttatttggagtttccactaaaatagtacaattgtatttttatacggcttttctgttgattccataagacttgttacttcgctgtatgattttctaacgatttttgttgttgttttgttggaactcttattaaggttttacaaaattacattatacatgtacaaatcattcagtgcaatctccatttaagaatcgtttcttcaagtaaactcttggttataatgataatgtttttataactagtgttcttttgaagatccaatgcgttgtcgttccagtagtggggaagtaaaagtccccagtagtggggaagtccccaaattctgcgattggtatattactttttaattaatattcatttgccatacgtctacatcttattaaaatatgcctttatatgatctataattactggtattatataaattcataatagttatcatctgtttttaattaggtttattcttaatacatattcataatatgcctaataaaaaatcccaccatgttgatcccgcgtaggacctattattttttaagaaaccggttaccaaaactatcaaatagtagtcggtccttaagggtttcttacactgttcgcaactctcactgacacgtggcggtccgcgattggttcgttttattttatttttttctaaatttgaaaaagtgaaaaaaaaaattaagaaaaaaaattaagaaatctgggataatgatggtcttagtatagaaatttcttaatcatggacgaatgaaaagagctcctgagttcattttttcagagagatatctagctaggatttttttggtcgaactttgaatggtttttgttcttgatgccgctaaagattatatgattctgtaaattaatggactcagttattgattagtttaagacactagtaacataaaccatattagttataaatgtcttaagacaacacgagagagagcgcttgcccctaaagtgtctctctctctcccaagttggaaaacccctataaatagatgtcttggtcatctttgttaaccca

SEQ ID NO: 76 - DNA, 2000 bp, Arabidopsis thaliana
  source 1..2000: mol_type = genomic DNA
gtgaccaggcttctcgtatttgagttctatggctcttacttgccaaaagagcttccttcgtcaaagaggtgttttggggtcgccaaagttaataaccagaggccggggatagagaagattcttttgggggggtttggggtcgtgactcatgtcgtaagttgttgcttgctacatcttttggattaagcttcacctaagctaaatagctagttgatacgttgtctactttttttttttttttgtcgttgatacgttgtctacttgtattgagcacattgatgtttctcttttctttttgaaaattcgatttatctttttttttttttctaaattgttaaagggttgtcttgtttggagaatttgttgtagacaactttatataagtttgatatattttgctttgtgcctttattctgattaattaccaaatcttaataaagcatcaatcaagactttctaatataagaaaataaataaattattatttagtggagaatccataataagttaataatcattatattcaataggacaaaacctttgggtgtgtttatattagtctacggattttctgatttgtataacacttttggtatttcagattaatagagtattatggttgaaatatgcattcgtaaaatcttcggaagttttaaggttttgtaactaataatcatatatagaatcccaatttgaagaatcggttcttcacgttggttttattgattcgataaaaagaagtttttttaagaatcaatgcgacaatgcgttgtcgttccacgagcaaggagtcccaaatctgcgatatgtttctttcaaatagcctctgagctaggcctgggcattcagtttcggttcggttgcgcatggtttcggttatttcggttatagtaatataagaaccatttggatatttgaggaattttggttcggttcggttcggatatctttcggttcggttcggtttggatattaaacttggtaaccataaataaactaaaaccgaaataaccgactaaaaaccgaaacaaccaaaaaatttgaccaataaccgatatagaacgtacgtaaaaaagagaaatattcacaactcacaaagatggtccatactctccataatattaaaacacctgattcacaactcacgaaattcaacttgcaaactaataaagagaaaaacatagttaagtgtcaatacatactgtgatatattatctatcatacatatatgctttggttttcatttggttatcggttattaacctaaccgaaaccgaaaccgaaatctaagacatataatattcaaccggttattttaagctatccaaacctgaaccgaaccatgtttttcggttcgattcggttcggttaatcggttagcggtttttttgcccaggcctactctgagcaataattgtatccggagttgtaatagaatcaaagtacgatgagagtgtttttatgacaaatatcttaatcttggccaattatatgttctactgaaattctttttgaattcatcgaccagtgagacttaaaaatagctttttattcgccgaggtatatatagctaggaattttgtcgaaatttagacgttagtgggttttgttcttcgtgacacaaaagatattctatatattaacgaaatctagcgatcgatatggtatttatataaagtcttggtcatagataggggttgaaacttgaaaccatgcatgatatgccaatgttgctgaagcagtcaatgttgctgaagaagtcaaacgtaattatatagtgaataccaaaaaagtgatatttcttaattcaattaaatataattatagttttaaatcacctaaaataagttacttattaaaaccccccaaatttactttaatatagttggtgtacatgtttgagaaagcaaacaaaaagaaaaagaaaaagaaaaaaaaaagagaaagaggttagtacacataattgggaattaatgtctattgattcttttatctttctc

SEQ ID NO: 77 - DNA, 2000 bp, Medicago truncatula
  source 1..2000: mol_type = genomic DNA
tcacattttgtttccacctttctcttatttttgggactcataaagcagagatcaaaattgtcagtgaatttttttttaggattaaaggtcgaagaatttttttatggagactaaaaccaaaagtttgaatatttatagggacaaaaaacatatttaactataattaaaaatattaaaaaataaatacattaaaaaatgtgtgtgtctatatgtatattaaaaaaaaaaaaacatgtccttttatgtcattttatacttatcaaccacttcaacagctaattttaccaaacacattatttttaataagcaagcttttcagctataagctatcagctaccagctatcagctagcttatggcttctttttaccaaacagagcctatgtgtttagcaatgagtttttctatattttctttgttccttttcttattaccttgctctaaattcatataatataattataattttacataaaatatacaataagtagtgaagaaaatgtttagtaaaaaaaaatgttaattgaattcataaaacacatttatagttttgaagatgaaagtaatttgaatatttaaaaagaaataagttaagtaaaaaaaattatgtatataatttaaaaaaaaaaataaacatgtctttttttgtcattttatatgtatcagtcatttcaacagttaattttatcaaacacattatttttaataagcaagcttttcagctataagctatcagtcatcagctataagctagcttttcagttatcagccagtttatagcttctttttaccaaacagagccttaatcaggtttgaatttgggtcatcgtatttctgctctatgcatttttattttattatgtgttgaccactaaaacaatagaaaagagaaagagagagggacaaaaattgattcactaatttcctaattgatgttggataaaaatacctgttcgaaaaggttaatctcttaagagttttacattggatgagagatgacatgaacgtatgtttataaaagggagtgaactcaacttacaagccagttttgtaaggttaagttagaccccacctaaattataagatgatattaaagtttatcaaaaatcatttgggtcatatgctatcaggtttctactatcagtccacttgaatcatgctccatatatatagttttgggcgtgagagggtatgtcaagagtttcacgttaaataagagatgacaaacatatatttataaatatagataattcttcctttacagactaattttgtaaggttgagatacatacacccgactaaaatagttagataatctctcttaagttaatcatgtgttttacttcatgaatagtctccacaatgtgcatataccttattagaagggaaaaaaacatatgatatctggtcaattgttattttttaaagaaaataatcaatactaatcaattcatggagtttttatccgtttgttagaatttaaacaaaaaatctatatctacttatatatatatatatatatatatatataaacttgtttcaaattataatattttttagtataaattctcttaaaatctcattttcgtacatgacttacaaacatataatttatgacaaacaaaaacccaaaataacttatgtttttcttattgtaaaaaaactcatattattctaaagtttttttaagaataatcaataaaatattaaaaagttttagattatgccaatttgctaggaattattccccattgtgtacgtgtaggggagagacaaagagagtaaaaggggtttgtttgatttgtcatctaatgtaacatgactaacatgaaacattcctgagggactagctagttgaaatgaacagaaccaaaccaatatcaagagtggtgggagcacctgaacctgtatttttacaccctatagaatccctcttttctatagatctaattaaattatatcttgcatgtaacatatttaaaatgggaaaaataacactta

SEQ ID NO: 78 - protein, 175 aa, Arachis hypogaea
MARIMSSEPLIVGRVVGDVLDPFNASIRMSVSYGNRQVYNGHEFFPSTVTFKPKVEIGGTELRPFFTLVMTDPDVPGPSDPYLREHLHWIVTDIPGTTDATFGKELVSYEMPKPNIGIHRYVFVLFKQKRRQCVSTIPSCRDHFNTRTFAADNDLGLPVAAVYFNAQRETAARRR

SEQ ID NO: 79 - DNA, 2000 bp, Arachis hypogaea
  source 1..2000: mol_type = genomic DNA
ttaacaatcatctaaaacaccgtcaactcaaaataaaccctaaattataccctacctaacctaatattatttagtttctaattacactaaattaatataacaaaccctaatcacacacttctttaaaatttttaatcaataatttaataaaatacctaacaaaattctaaataaaaaatctgaaaaaatacaaaaattataaaaaaaaaattacctttgatggtggctacaaaatagtgaaagtgtagtggtgacagaagacaacagtaaaacctatagtggtagagctaaagtttagtaacataacaaacaaccaaaaaaaatggagaacaaccttactttggccggtagacctcagtcacggtgatggcgacgacctccaatcgatggcaagggacaccaacagcagcaacacgacggctttccacaaagactccaacagcagtgacgatctgcgacgacaatgttgcttaccagtggcgacggcacaccctttttcattggaccaaagggagagagatgagagagaagaagagaagagagagagaaagagaaagagtttgcaaaaataaggaagaatgatttcacgtttaaattttatcccaaatttatcattggatttaccggcagaaaaaaccgacaataaaatgttcatcgttgtctaaacgatgcatttcgataaatcttgttactatcggaattttctgacggaaaatccgacgataaatttggcgctaataaaattttcgcacctctatttctatcggatttagcggcgaaattttaaatccgacaataacttattcaatggacaaatttttaatcatatctgtcaaaaatttcattattaaatccaacgataagtcaccaaaaaaaaaatccaacgataacgtctattgctatatttgtcagtaaatctaacgatactcaatattttttatagtgtacgttgatgcaatatttttattttttatttatccaatatctgaatatatagcgcaaatataaactaggtttgaactttcgatcaattttaagggtgacaattgtaaaataaattcaataagttattcctactctatcgcactacaataaaaatttcttttaattggtaaatttttagtagctataatataatatataattattatatattttatttaatttatatttttgtgataattatatatttttatatcattaaataattattgttaaaattttttaacgataaaatataaaataattaatatttaattgtcaataaatatactaataattatttttattgttattaaaaacaaaataaattacctctaaaactttttttttttagtagcatcgttagtggggtgatttgaaaaaactcctctgaaccgaaaaaaaatcgtaagataataaaatagcgaattaatcatcattaaattttttatttatattatgagtgattttttctattttgatctaacaatatgattagatacttaattaattttttattttaccaatttttatataacattagaaaaaaaattacataagtgattgatcaaatataactagcgctatacaaaatggatagtaaaagcaaaaaggacgaagtttaatttgtcccgcataacatcataagatgagtagaataacgatttaaagagaaacatatcggagagacacttctcttgcaatgaggcaaattaaagagacagcattttatgtgaggagtggtgggagcatatgatttgtttcagccctcatcgacccaattattagaataaacaaatgatatcttagtgtgtagtaacacatgtattcacaaaaatgaatattaaaaacacatagctggaagattctcttgatagagacattataaatacggaactcgagtgtacttactttcctcaagctaagctagtaattgtatttgtatctctcactctttattaatttcctctatcttcttcttatttgcttgttaataagaattatattattctcctaat

SEQ ID NO: 80 - protein, 174 aa, Cicer arietinum
MARMSQEPLLVGRVIGEVLDSFTTSMKMTVSYNKKQVFNGHEFFPSTINIKPKVEIDGGDMRSFFTLVMTDPDVPGPSDPYLREHLHWIVTDIPGTTDATFGKELVSYEIPKPNIGIHRFVFVLFKQKSRESVMTTPSSRDHFNTRNFASQNDLGLPVAAVYFNAQRETAARRR

SEQ ID NO: 81 - DNA, 1997 bp, Cicer arietinum
  source 1..1997: mol_type = genomic DNA
aaagaaagaagaataacaccctctaaacagaaaattgcattttgggtgcatgcaattaaggaaggttctctacgcaagccattcaattaaaattaaaaaataaaattatccatgtccataatatgctactattttgccttatatgctaaagaacatggtaaattaatgtggacgttgaagaaacaaaagctagcatttcctagaattttaatttgccttttttatatgcttttagtttttaataaaaataaataaaaaatggtagagactgacaactagcaaaagatggaccccacatagagtttaagacacatgatgctttttgtgagctgggaagaaaactcccttttattggtcgacacaaattcattcatccaatcaagaaccgattccaagtgacccgtcatggagcattttctttttccatcaattcggtgtttatttgcacagttacaatttcatttttaaaatatactcataactatttaattttctaaattttatataaaatataaattaaatttagtaaataatttttttaatgttaaatatttaactattaacattaaatactttaattaaatgaataatatattaagaataataacattaaataattcaaaaataattaactgttacttataataataatcaagatttcgtattattttatgtgattataacagtgaaaagaggaaatataactaacataatgaataagaacattaaatctacactatacataataacattaatatggtgtgacaatatttaaattacttaattgtttgattgaaattcgaactttaacggatacacgtgaaaagatatatattagaagaagttatcatcataatcgaaacattcaattctgccatctcgcacaaagggatatatatctagtttataaaaaaataaaataaaataactatgtattatttaaacgagtaaattaaccgttattttaggatatgaaattttagactctagtcaattaatttgtcagctcctatttatgaaatatcaaattatttaaaattaaataaaatgtttcttttgtcaattattttatagaattttttttctacgagaagggacagatacattcgttcctggactttttttttttgtttaatatactttaaaacattttactcttatataaaaaaatagttgttgcctccaccatatttttttaaaatttgttcattatatttatatctttacgcattccatgtgttctttcttaattttagaggatcaaacattttttatatgaaaatattatagattattaatatttttaatttctaaaaccaattaagtttgagcctttattctatatttttgttctatagaaagtattatactttttttttttttttacctccaataaacaatatttttagatccgccattgtatataagttaaaagtccaagaagtattttgaccgacattaaacaatttaaaaaaataatttgatattttataaatttaatttgatatactaaaaaatatattgtaatatgtgtgacttaataataagttaataataagtatgaatgattgatagtgtaaattttaatttacaatgaatgaaaagttgcaagaataattccccattttgtacatgtagggagagagataaagagaggaaaagggattggattagtcatgtaacatgactaacatgatttcagcataaactttagtgagtgactaaactaggtagttcaaatgaaccaaaccaatatctcaaagtggtgggagcacctcaaccttattttattttatttttacactccatagagttcctctttttctctatatctaattaaattatgtgttaattgcaacatgtatattttaaacttgaaagatactttttttaagacattataaatatggagttctcaataacttgttttctcatcaacaagaattatttgagttgtactcactcttgagtttcattattttctctttttcttcaaa

SEQ ID NO: 82 - DNA, 1995 bp, Glycine max
  source 1..1995: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgaccgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 83 - DNA, 1971 bp, Glycine max
  source 1..1971: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 84 - DNA, 1962 bp, Glycine max
  source 1..1962: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 85 - DNA, 1913 bp, Glycine max
  source 1..1913: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaaaggaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 86 - DNA, 1951 bp, Glycine max
  source 1..1951: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 87 - DNA, 1989 bp, Glycine max
  source 1..1989: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 88 - DNA, 1972 bp, Glycine max
  source 1..1972: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 89 - DNA, 1989 bp, Glycine max; identical to SEQ ID NO: 87
  source 1..1989: mol_type = genomic DNA

SEQ ID NO: 90 - DNA, 1959 bp, Glycine max
  source 1..1959: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaatatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 91 - DNA, 1765 bp, Glycine max
  source 1..1765: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctctgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 92 - DNA, 1980 bp, Glycine max
  source 1..1980: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgcaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 93 - DNA, 1663 bp, Glycine max
  source 1..1663: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctctgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 94 - DNA, 1839 bp, Glycine max
  source 1..1839: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 95 - DNA, 1983 bp, Glycine max
  source 1..1983: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcactatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 96 - DNA, 1717 bp, Glycine max
  source 1..1717: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 97 - DNA, 1880 bp, Glycine max
  source 1..1880: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 98 - DNA, 1918 bp, Glycine max
  source 1..1918: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgcttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 99 - DNA, 1940 bp, Glycine max
  source 1..1940: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcatactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 100 - DNA, 1993 bp, Glycine max
  source 1..1993: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtcctagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 101 - DNA, 1814 bp, Glycine max
  source 1..1814: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 102 - DNA, 1984 bp, Glycine max
  source 1..1984: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctggtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 103 - DNA, 1941 bp, Glycine max
  source 1..1941: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctggtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 104 - DNA, 1859 bp, Glycine max
  source 1..1859: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcaactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgaccaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 105 - DNA, 1990 bp, Glycine max
  source 1..1990: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcaactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 106 - DNA, 1432 bp, Glycine max
  source 1..1432: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgaccaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 107 - DNA, 1974 bp, Glycine max
  source 1..1974: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 108 - DNA, 1889 bp, Glycine max
  source 1..1889: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 109 - DNA, 1337 bp, Glycine max
  source 1..1337: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 110 - DNA, 1902 bp, Glycine max
  source 1..1902: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 111 - DNA, 1966 bp, Glycine max
  source 1..1966: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 112 - DNA, 1921 bp, Glycine max
  source 1..1921: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 113 - DNA, 1939 bp, Glycine max
  source 1..1939: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 114 - DNA, 1619 bp, Glycine max
  source 1..1619: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgtttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 115 - DNA, 1813 bp, Glycine max
  source 1..1813: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcactcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 116 - DNA, 1886 bp, Glycine max
  source 1..1886: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacggcagaagcttgacaacttgcaaaggggagggaggacccacacacacaggattacacatgatgctttttttgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 117 - DNA, 733 bp, Glycine max
  source 1..733: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaa

SEQ ID NO: 118 - DNA, 1810 bp, Glycine max
  source 1..1810: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 119 - DNA, 1993 bp, Glycine max
  source 1..1993: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 120 - DNA, 1957 bp, Glycine max
  source 1..1957: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 121 - DNA, 1954 bp, Glycine max
  source 1..1954: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 122 - DNA, 1995 bp, Glycine max; identical to SEQ ID NO: 82
  source 1..1995: mol_type = genomic DNA

SEQ ID NO: 123 - DNA, 1045 bp, Glycine max
  source 1..1045: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 124 - DNA, 1962 bp, Glycine max
  source 1..1962: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 125 - DNA, 1876 bp, Glycine max
  source 1..1876: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacggcagaagcttgacaacttgcaaaggggagggaggacccacacacacaggattacacatgatggcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 126 - DNA, 1542 bp, Glycine max
  source 1..1542: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 127 - DNA, 1919 bp, Glycine max
  source 1..1919: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

SEQ ID NO: 128 - DNA, 1911 bp, Glycine max
  source 1..1911: mol_type = genomic DNA
attgaattttttaagaaacaattttaaattatatagttatacataaataaacaaaatacaaaaaaaacaaaaatcaaatcatgtgaagattttcaaaaaaatacatgacttttagaaaaaatcacaaaacttgcatgtatgagagaaagagacgagggttaattaatttaacttcttcaaaattttctttttattttttctgtatgtatgcatatattatttgaaaatttaattgaaatacactgataatataaggattttttatactgttgtttaatcataaaacatcatatagtgaaaatttgttaaattttatacaataattattttaaaaaattatcttaaaaaattactttaaaaaaatacatttttggtacatataaaatttatactaacagtatataaaaattaagctaaatataaaaagtatatttaaaaaaattatgttgtatataataccaatatgaccaacgaatattccatgcttaatcggcatcactaacttttaatcaagttaaacgaagaaagtaggaactaaaagataaacatgagcaacttagcattttaattataatgatattggaagtttaggaaagaaaacctaaaatatccaaatttagaaatatgaatatcaacttataccaaaataagcaactaacagtttcagaccaacaaattttaaaatcatcatctccatatgccacataattaataattaagtctcaaattgaaataatattatattttcacagtgaaacgataccaacctgcaatttatttatgcaataaacggtctatatattccactttcaaatcataatataaaaaaatcaatataacgaatgtctgcttaattaattatacgtactaattaaactatgccaccaccaattagggcttttttattttctggaactagaatttgtgatcttttaagcacgggatgtgcatgtacacacacctcaacccctttttctccaacaaaagaatgcaaacagaacttgaataacataaaatttcacatgcagagatgaagagggcaaaatggtttcttgataaagaaggtccaccttcctcttttaccaatattggaacttggtctgcatctaagagttaagtttttattctgaaatgacctcttaggcactcaaacgcttatactccataggcgtgtctagccttgagaatttgtggtggagttcctttaattatgccctcttcatcaggaatcttccaacaaaattaaaagaaagactatggctaagggaaaatggagaatcacgcacgcacaaacagaaatttgcatagtgtgtgcaaacaattaaggaaggttctctacgcaaggcatacaacagaaatttctgcacgtccatggtactagtagtatactaaagagaatgctaaaatgtgggtgttcaagaaacgaagcatttcctagaatttttgccttttatgcttttagtttaataataaaacggcagaagcttgacaacttgcaaaggggagggaggacccacacacacaggattacacatgatgctttttttggaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctc

(19) United States
(12) Patent Application Publication
(10) Pub. No.: US 2023/0235350 A1
(43) Pub. Date: Jul. 27, 2023

(54) COMPOSITIONS AND METHODS FOR ALTERING PLANT DETERMINACY

(71) Applicant: Monsanto Technology LLC, St. Louis, MO (US)

(72) Inventors: Brent Brower-Toland, St. Louis, MO (US); Jeongwoon Kim, Chesterfield, MO (US); Keith Merrill, O'Fallon, MO (US); Linda Rymarquis, High Ridge, MO (US); Thomas L. Slewinski, Chesterfield, MO (US); David R. Wooten, JR., Middletown, DE (US)

(21) Appl. No.: 18/053,629

(22) Filed: Nov. 8, 2022

Related U.S. Application Data
(60) Provisional application No. 63/278,903, filed on Nov. 12, 2021.

(51) Int. Cl. C12N 15/82; A01H 6/54; A01H 5/10; C12N 9/22

(52) U.S. Cl. CPC C12N 15/827; A01H 6/542; A01H 5/10; C12N 9/22

(57) ABSTRACT

Provided are compositions and methods for altering TFL1 levels in soybean plants. Methods and compositions are also provided for altering the expression of genes related to the timing of terminal differentiation of stem tips through suppression, mutagenesis and/or editing of the TFL1 gene. Modified plant cells and plants having a suppression element or mutation reducing the expression or activity of a TFL1 gene are further provided comprising reduced TFL1 levels and improved characteristics, such as reduced plant height and increased lodging resistance.

### INCORPORATION OF SEQUENCE LISTING

This application claims the benefit of U.S. Provisional Appl. Ser. No. 63/278,903, filed Nov. 12, 2021, the disclosure of which is incorporated herein by reference in its entirety.

### INCORPORATION OF SEQUENCE LISTING

The sequence listing that is contained in the file named “MONS522US_ST26.xml,” which is 265 kilobytes as measured in Microsoft Windows operating system and was created on Nov. 7, 2022, is filed electronically herewith and incorporated herein by reference.

### FIELD OF THE INVENTION

The present disclosure relates to the field of agricultural biotechnology, and more specifically to methods and compositions for genome editing in plants.

### BACKGROUND OF THE INVENTION

Precise genome editing technologies are powerful tools for engineering gene expression and function and have the potential to improve important agricultural traits. A continuing need exists in the art to develop novel compositions and methods to effectively and efficiently edit the plant genome in various crop plants in order to alter determinacy, increase yield, reduce lodging, and achieve other beneficial results.

### SUMMARY

Provided herein are modified plants, plant seeds, plant parts, or plant cells, comprising a modification that reduces the expression or activity of TFL1, or a homolog thereof, as compared to the expression or activity of TFL1 or the homolog thereof in an otherwise identical plant, plant seed, plant part, or plant cell that lacks the modification. In some embodiments, the modification is present in at least one allele of an endogenous TFL1 gene or homolog thereof. The TFL1 gene can be a TFL1b gene. In particular embodiments, the TFL1 gene or homolog thereof encodes a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2. In other embodiments, the modification is in a non-coding region of the TFL1 gene or homolog thereof, non-limiting examples of which include a promoter, an intron, a 5′-untranslated region, a 3′-untranslated region, and combinations of any thereof. The plant may be, or a plant seed, plant part or plant cell provided herein may be from, for example, a leguminous plant, a cotton plant, a canola plant, a corn plant, a sorghum plant, a rice plant, a wheat plant, a barley plant, a tomato plant, or a pepper plant, or other crop, ornamental or other type of plant species. In further embodiments, the leguminous plant is a soybean plant, a bean plant, a pea plant, a chickpea plant, an alfalfa plant, a peanut plant, a carob plant, a lentil plant, or a licorice plant. In specific embodiments, the leguminous plant is a soybean plant. In some embodiments, the plant, plant seed, plant part, or plant cell is heterozygous for the modification, and in other embodiments, the plant, plant seed, plant part, or plant cell is homozygous for the modification. In certain embodiments, the plant, plant seed, plant part, or plant cell is defined as comprising a first modification in a first allele of the TFL1 gene and a second modification in a second allele of the TFL1 gene, the first modification and the second modification being different from one another.

A modified plant, plant seed, plant part, or plant cell provided herein may, in certain embodiments, comprise a modification that reduces the expression or activity of TFL1, or a homolog thereof, wherein the modification comprises a deletion, an insertion, a substitution, an inversion, or any combination thereof. In some embodiments, for example, the modification is located at about 100, 125, 150, 175, 200, 225, 250, 275, or 300 nucleotides or more from the 3′ end of a sequence selected from the group consisting of SEQ ID NOs:4, 67-77, 79, and 81. In other embodiments, a modification in the promoter of at least one allele of the TFL1b gene is comprised within a genomic region between nucleotide positions 1237 and 1570 of reference sequence SEQ ID NO:4. In some embodiments, the modification comprises a deletion of at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 125, or at least about 150 consecutive nucleotides. The plant, plant seed, plant part, or plant cell can also comprise, for example, a modification in at least one allele of a promoter of the TFL1b gene, wherein the modification is selected from the group consisting of: a 30 base pair deletion from nucleotide 1539 to nucleotide 1568, as compared to reference sequence SEQ ID NO:4; a 388 base pair deletion from nucleotide 1217 to nucleotide 1604, as compared to reference sequence SEQ ID NO:4; a 112 base pair deletion from nucleotide 1518 to nucleotide 1629, as compared to reference sequence SEQ ID NO:4; a 272 base pair deletion from nucleotide 951 to nucleotide 1222, as compared to reference sequence SEQ ID NO:4; a 44 base pair deletion from nucleotide 1364 to nucleotide 1407, as compared to reference sequence SEQ ID NO:4; a 150 base pair deletion from nucleotide 1367 to nucleotide 1516, as compared to reference sequence SEQ ID NO:4; a 1053 base pair deletion from nucleotide 754 to nucleotide 1806, as compared to reference sequence SEQ ID NO:4; a 104 base pair deletion from nucleotide 1216 to nucleotide 1319, as compared to reference sequence SEQ ID NO:4; an 807 base pair deletion from nucleotide 1159 to nucleotide 1965, as compared to reference sequence SEQ ID NO:4; a 455 base pair deletion from nucleotide 760 to nucleotide 1214, as compared to reference sequence SEQ ID NO:4; a 90 base pair deletion from nucleotide 939 to nucleotide 1028, as compared to reference sequence SEQ ID NO:4; a 37 base pair inversion, wherein the sequence from nucleotide 1029 to nucleotide 1065 of SEQ ID NO:4 has been deleted, inverted, and reinserted at the same location; a 599 base pair deletion from nucleotide 1066 to nucleotide 1664, as compared to reference sequence SEQ ID NO:4; a 601 base pair deletion from nucleotide 952 to nucleotide 1552, as compared to reference sequence SEQ ID NO:4; a 132 base pair deletion from nucleotide 1677 to nucleotide 1808, as compared to reference sequence SEQ ID NO:4; a 35 base pair deletion from nucleotide 1524 to nucleotide 1558, as compared to reference sequence SEQ ID NO:4; a 930 base pair deletion from nucleotide 476 to nucleotide 1405, as compared to reference sequence SEQ ID NO:4; a 195 base pair deletion from nucleotide 1365 to nucleotide 1559, as compared to reference sequence SEQ ID NO:4; a 633 base pair deletion from nucleotide 928 to nucleotide 1560, as compared to reference sequence SEQ ID NO:4; a 1221 base pair deletion from nucleotide 593 to nucleotide 1813, as compared to reference sequence SEQ ID NO:4; a 5 base pair deletion from nucleotide 1552 to nucleotide 1556, as compared to reference sequence SEQ ID NO: 4; a 29 base pair deletion from nucleotide 1537 to nucleotide 1565, as compared to reference sequence SEQ ID NO: 4; a 38 base pair deletion from nucleotide 1209 to nucleotide 1246, as compared to reference sequence SEQ ID NO: 4; a 49 base pair deletion from nucleotide 1552 to nucleotide 1600, as compared to reference sequence SEQ ID NO: 4; a 49 base pair deletion from nucleotide 1553 to nucleotide 1601, as compared to reference sequence SEQ ID NO: 4; an 11 base pair deletion from nucleotide 1368 to nucleotide 1378, as compared to reference sequence SEQ ID NO: 4; a 28 base pair deletion from nucleotide 1368 to nucleotide 1395, as compared to reference sequence SEQ ID NO: 4; a 41 base pair deletion from nucleotide 1344 to nucleotide 1384, as compared to reference sequence SEQ ID NO: 4; a 235 base pair deletion from nucleotide 1219 to nucleotide 1453, as compared to reference sequence SEQ ID NO: 4; a 13 base pair deletion from nucleotide 1215 to nucleotide 1227, as compared to reference sequence SEQ ID NO: 4; a 7 base pair deletion from nucleotide 1370 to nucleotide 1376, as compared to reference sequence SEQ ID NO: 4; a 337 base pair deletion from nucleotide 1220 to nucleotide 1556, as compared to reference sequence SEQ ID NO: 4; a 161 base pair deletion from nucleotide 1216 to nucleotide 1376, as compared to reference sequence SEQ ID NO: 4; a 17 base pair deletion from nucleotide 1368 to nucleotide 1384, as compared to reference sequence SEQ ID NO: 4; a 283 base pair deletion from nucleotide 1366 to nucleotide 1648, as compared to reference sequence SEQ ID NO: 4; a 120 base pair deletion from nucleotide 1370 to nucleotide 1489, as compared to reference sequence SEQ ID NO: 4; a 75 base pair deletion from nucleotide 1541 to nucleotide 1615, as compared to reference sequence SEQ ID NO: 4; a 9 base pair deletion from nucleotide 1367 to nucleotide 1375, as compared to reference sequence SEQ ID NO: 4; a 51 base pair deletion from nucleotide 1551 to nucleotide 1601, as compared to reference sequence SEQ ID NO: 4; a 7 base pair deletion from nucleotide 1371 to nucleotide 1377, as compared to reference sequence SEQ ID NO: 4; a 186 base pair deletion from nucleotide 1368 to nucleotide 1553, as compared to reference sequence SEQ ID NO: 4; a 14 base pair deletion from nucleotide 1365 to nucleotide 1378, as compared to reference sequence SEQ ID NO: 4; a 16 base pair deletion from nucleotide 1365 to nucleotide 1380, as compared to reference sequence SEQ ID NO: 4; a 43 base pair deletion from nucleotide 1552 to nucleotide 1594, as compared to reference sequence SEQ ID NO: 4; a 10 base pair deletion from nucleotide 1367 to nucleotide 1376, as compared to reference sequence SEQ ID NO: 4; a 131 base pair deletion from nucleotide 1552 to nucleotide 1682, as compared to reference sequence SEQ ID NO: 4; a 438 base pair deletion from nucleotide 954 to nucleotide 1391, as compared to reference sequence SEQ ID NO: 4; a 26 base pair deletion from nucleotide 1535 to nucleotide 1560, as compared to reference sequence SEQ ID NO: 4; a 111 base pair deletion from nucleotide 1534 to nucleotide 1644, as compared to reference sequence SEQ ID NO: 4; a 663 base pair deletion from nucleotide 937 to nucleotide 1599, as compared to reference sequence SEQ ID NO: 4; a 98 base pair deletion from nucleotide 1551 to nucleotide 1648, as compared to reference sequence SEQ ID NO: 4; a 34 base pair deletion from nucleotide 1526 to nucleotide 1559, as compared to reference sequence SEQ ID NO: 4; a 79 base pair deletion from nucleotide 1528 to nucleotide 1606, as compared to reference sequence SEQ ID NO: 4; a 61 base pair deletion from nucleotide 1542 to nucleotide 1602, as compared to reference sequence SEQ ID NO: 4; a 381 base pair deletion from nucleotide 1214 to nucleotide 1594, as compared to reference sequence SEQ ID NO: 4; a 187 base pair deletion from nucleotide 1368 to nucleotide 1554, as compared to reference sequence SEQ ID NO: 4; a 109 base pair deletion from nucleotide 1369 to nucleotide 1477, as compared to reference sequence SEQ ID NO: 4; a 5 base pair deletion from nucleotide 1550 to nucleotide 1554, as compared to reference sequence SEQ ID NO: 4; a 1267 base pair deletion from nucleotide 734 to nucleotide 2000, as compared to reference sequence SEQ ID NO: 4; a 190 base pair deletion from nucleotide 1371 to nucleotide 1560, as compared to reference sequence SEQ ID NO: 4; a 36 base pair deletion from nucleotide 1537 to nucleotide 1572, as compared to reference sequence SEQ ID NO: 4; a 46 base pair deletion from nucleotide 1541 to nucleotide 1586, as compared to reference sequence SEQ ID NO: 4; a 5 base pair deletion from nucleotide 1552 to nucleotide 1556, as compared to reference sequence SEQ ID NO: 4; a 955 base pair deletion from nucleotide 669 to nucleotide 1623, as compared to reference sequence SEQ ID NO: 4; a 38 base pair deletion from nucleotide 1521 to nucleotide 1558, as compared to reference sequence SEQ ID NO: 4; a 109 base pair deletion from nucleotide 1369 to nucleotide 1477, as compared to reference sequence SEQ ID NO: 4; a 15 base pair deletion from nucleotide 1540 to nucleotide 1554, as compared to reference sequence SEQ ID NO: 4; a 458 base pair deletion from nucleotide 1217 to nucleotide 1674, as compared to reference sequence SEQ ID NO: 4; an 81 base pair deletion from nucleotide 1546 to nucleotide 1626, as compared to reference sequence SEQ ID NO: 4; an 89 base pair deletion from nucleotide 1547 to nucleotide 1635, as compared to reference sequence SEQ ID NO: 4; and combinations of any thereof. In some embodiments, the plant, plant seed, plant part, or plant cell comprises a polynucleotide sequence selected from the group consisting of SEQ ID NOs:30, 31, 32, 33, 34, 35, 36, 37, 38, 39, 40, 41, 42, 43, 44, 45, 46, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, and 128. A plant, plant seed, plant part, or plant cell provided herein can also comprise, for example, a chromosomal sequence in the TFL1b gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO:4 in the regions outside of the deletion, the insertion, the substitution, or the inversion.

Also provided herein are modified plants or the seeds, plant parts, cells thereof, comprising a modification that alters the determinacy phenotype of the plant relative to an otherwise identical plant that lacks the modification. In certain embodiments, the modification increases the determinacy of the plant, as compared to the determinacy of an otherwise identical plant that lacks the modification. In some embodiments, the modified plant reaches its terminal flowering date sooner, exhibits a reduced lodging rate, exhibits substantially the same or increased yield, or exhibits lower susceptibility to fungal disease, or any possible combination thereof, as compared to an otherwise identical plant that lacks the modification.

In certain embodiments, a polynucleotide is provided comprising a sequence selected from the group consisting of SEQ ID NOs:30, 31, 32, 33, 34, 35, 36, 37, 38, 39, 40, 41, 42, 43, 44, 45, 46, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, and 128 is provided. Also provided is a guide RNA comprising a polynucleotide sequence selected from the group consisting of SEQ ID NOs:13, 14, 15, 16, 17, 18, 19, 20, 21, 22, 23, 24, 25, 26, 27, 28, and 29.

Further disclosed herein is a method for producing a plant comprising a modified TFL1 gene, the method comprising: a) introducing a modification into at least one target site in an endogenous TFL1 gene or a homolog thereof of a plant cell; b) identifying and selecting one or more plant cells of step (a) comprising said modification in said TFL1 gene or homolog thereof; and c) regenerating at least one plant from at least one or more cells selected in step (b). In some embodiments, the target site is located in a non-coding region of an endogenous TFL1 gene or homolog thereof. In other embodiments, the non-coding region is selected from the group consisting of a promoter, an intron, a 5′-untranslated region, a 3′-untranslated region, and combinations of any thereof. In further embodiments, the non-coding region is a promoter. In still further embodiments, the modification is facilitated by the presence of at least one site-specific genome modification enzyme in said plant cell. Non-limiting examples of such an enzyme include an RNA-guided nuclease, a zinc-finger nuclease, a meganuclease, a TALE-nuclease, a recombinase, a transposase, and combinations of any thereof. Examples of RNA-guided nucleases include a Cas nuclease, a Cpf1 nuclease, or a variant of either thereof. Some site-specific genome modification enzymes that could find use in accordance with the disclosure create at least one strand break at the target site. The methods disclosed herein may be used, for example, to produce any modification in accordance with the disclosure, including a substitution, an insertion, an inversion, a deletion, a duplication, and a combination thereof. In some embodiments, the modification is a deletion and the deletion comprises a region of at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, or at least 150 consecutive nucleotides.

The methods provided herein may find use, for example, in the production of a plant having a desired phenotype. Non-limiting examples of such a phenotype include earlier terminal flowering date, reduced lodging rate, substantially the same or increased yield, lower susceptibility to fungal disease and increased determinacy, or any possible combination thereof. Such a phenotype may be defined, in specific embodiments, as being present in a plant when compared to an otherwise identical plant that lacks a modification that confers the phenotype according to the present disclosure.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the relative positions of the gRNAs on the approximately 2 kb promoter region upstream of the transcription initiation site (tis) of the GmTFL1b gene.

FIG. 2 shows unique, non-redundant homozygous deletions. Asterisks (*) indicate small deletions of less than 20 base pairs. Note that for the allele names, the prefix “AL” is omitted.

FIG. 3 shows the relative expression levels of the GmTFL1b gene in the shoot tip and leaf in plants comprising various Dt1 alleles generated through promoter editing. Expression of GmTFL1b in leaf tissue was nominal for all samples tested.

FIG. 4 shows images illustrating terminal raceme phenotypes. Photos were taken within two days after the terminal flowering date of wild-type plants.

FIG. 5 shows a pairwise comparison of the soybean TFL1b polypeptide sequence and the polypeptide sequences of putative homologs from relevant plant species identified through a BLAST search. The numbers with decimal points in the upper right triangle of the table show the percentage of identical residues in alignment positions to overlapping alignment positions between the two sequences. The integer numbers in the lower left triangle show the number of residue variations between the two sequences in the alignment positions.

### BRIEF DESCRIPTION OF THE SEQUENCES

SEQ ID NO:1 is the polynucleotide coding sequence of the Glycine max TFL1b (GmTFL1b) gene.

SEQ ID NO:2 is the amino acid sequence for the TFL1b protein (encoded by SEQ ID NO:1).

SEQ ID NO:3 is the polynucleotide sequence for the GmTFL1b gene, including the 5′ and 3′ untranslated regions (UTRs) and introns.

SEQ ID NO:4 is the polynucleotide sequence of the 2 kb promoter region upstream of the transcription initiation site (tis) of the GmTFL1b gene. SEQ ID NO:4 immediately precedes SEQ ID NO:3 in wild-type soybean plants.

SEQ ID NO:5 is the polynucleotide sequence of a common scaffold compatible with the Cpf1 gene.

SEQ ID NO:6 is the polynucleotide sequence for the Dahlia mosaic virus FLT promoter.

SEQ ID NO:7 is a polynucleotide sequence encoding a Lachnospiraceae bacterium Cpf1 RNA-guided endonuclease enzyme, codon-optimized for rice.

SEQ ID NO:8 is the polynucleotide sequence for a nuclear localization signal from Solanum lycopersicum.

SEQ ID NO:9 is the polynucleotide sequence for a Medicago truncatula ubiquitin promoter.

SEQ ID NO:10 is a polynucleotide sequence encoding a Lachnospiraceae bacterium Cpf1 RNA-guided endonuclease enzyme, codon-optimized for corn.

SEQ ID NO:11 is the polynucleotide sequence for a soybean RNA polymerase III (Pol3) promoter.

SEQ ID NO:12 is the polynucleotide sequence for a soybean 7SL_CR10 promoter.

SEQ ID NOs:13-29 are polynucleotide sequences for the spacer sequences in the guide RNAs (gRNAs) used for editing of the promoter region of the GmTFL1b gene.

SEQ ID NOs:30-46 and 82-128 are polynucleotide sequences for alleles of the GmTFL1b promoter having various deletions (and in the case of SEQ ID NO:40, also an inversion) as compared to SEQ ID NO:4.

SEQ ID NO:47 is the polynucleotide sequence of the 5′ UTR of the GmTFL1b gene.

SEQ ID NOs:48, 49, and 50 are the polynucleotide sequences for the first, second, and third introns, respectively, of the GmTFL1b gene.

SEQ ID NO:51 is the polynucleotide sequence of the 3′ UTR of the GmTFL1b gene.

SEQ ID NOs:52-66, 78, and 80 are the amino acid sequences of the TFL1b protein homologs found in Medicago truncatula (SEQ ID NO:52), Cajanus cajan (SEQ ID NO:53), Pisum sativum (SEQ ID NO:54), Arabidopsis thaliana (SEQ ID NO:55), Brassica napus (SEQ ID NO:56), Gossypium hirsutum (SEQ ID NO:57), Capsicum annuum (SEQ ID NO:58), Nicotiana tabacum (SEQ ID NO:59), Solanum tuberosum (SEQ ID NO:60), Solanum lycopersicum (SEQ ID NO:61), Zea mays (SEQ ID NO:62), Oryza sativa (SEQ ID NO:63), Hordeum vulgare (SEQ ID NO:64), Triticum aestivum (SEQ ID NO:65), Sorghum bicolor (SEQ ID NO:66), Arachis hypogaea (SEQ ID NO:78), and Cicer arietinum (SEQ ID NO:80), respectively.

SEQ ID NOs:67-77, 79, and 81 are the polynucleotide sequences of the approximately 2 kb promoter region upstream of the transcription initiation site (tis) of the TFL1b gene homologs found in Zea mays (SEQ ID NO:67), Sorghum bicolor (SEQ ID NO:68), Oryza sativa (SEQ ID NO:69), Triticum aestivum (SEQ ID NO:70), Hordeum vulgare (SEQ ID NO:71), Solanum lycopersicum (SEQ ID NO:72), Gossypium hirsutum (SEQ ID NO:73), Capsicum annuum (SEQ ID NO:74), Brassica napus (SEQ ID NO:75), Arabidopsis thaliana (SEQ ID NO:76), Medicago truncatula (SEQ ID NO:77), Arachis hypogaea (SEQ ID NO:79), and Cicer arietinum (SEQ ID NO:81), respectively.

### DETAILED DESCRIPTION

Stem growth habit is an important agronomic trait that directly affects plant characteristics such as plant height, flowering time and duration, node production, and root architecture in soybean (Glycine max). Plant height is an especially important agronomic trait in soybean and other crops, as it can directly affect yield potential and lodging resistance. Plant height is influenced by the timing of the transition from the vegetative phases to the reproductive phases at the shoot apical meristem. In soybean, growth habit and the timing of terminal differentiation of stem tips, both at the shoot apex and branch tips, is controlled by the Dt1 (indeterminate growth 1) locus. The Dt1 locus also influences other related traits, such as the branch density, stem pod density, stem node number, number of three-seed per pod, and total seed number. Wild-type expression of Dt1 specifies indeterminate growth and has incomplete dominance over the dt1 (determinate growth 1) allele, which causes determinate growth. Expression of the gene at the Dt1 locus, TERMINAL FLOWER 1b (GmTFL1b), at stem tips protects the apical meristem from terminal differentiation induced by FT2a, which is the soybean FLOWERING LOCUS T ortholog.

The majority of commercially cultivated soybean varieties, for example, are classified as having one of two stem growth habits, indeterminate or determinate. Indeterminate and determinate soybean plants are similar in their development during the vegetative growth phase, but have significant differences in stem growth habit at the reproductive/flowering stage. Indeterminate soybean varieties produce plants that grow in height from the tip of the stem for several weeks, while flowering simultaneously begins lower on the stem when the plant is still in the vegetative growth phase. In contrast, determinate soybean varieties produce plants that complete their growth in height on the main stem at the onset of the reproductive stages (R1 stage) and, at approximately the same time, produce all of the flowers that the plants will produce. As a result, determinate plants are only about one half to about two-thirds as tall as indeterminate plants.

Indeterminate soybean varieties generally have a higher yield potential compared to determinate varieties, but have the disadvantage of being more prone to stem lodging due to being more top-heavy. Because of its essential role in development, however, the Dt1 locus was fixed early in domestication of soybean, resulting in a general lack of diversity at the locus. This has inhibited efforts to develop varieties with novel determinacy phenotypes. Moreover, plant to plant competition in a soybean cropping context favors taller plants with greater flexibility, so that even in mutagenesis screens, the likelihood of selecting shorter, more compact semi-determinate plants with variation in degree of determinacy is very unlikely.

The present disclosure represents a significant advance in the art in that it provides engineered alleles that confer novel intermediate phenotypes between the current indeterminate and determinate growth habits (semi-determinate) in soybeans and other crops, as well as methods for the production thereof, thereby offering improvements in key traits that lead to reduced crop lodging and increased productivity per plant and plot. The methods and compositions disclosed herein offer the opportunity to create diversity that cannot be selected from conventional plant breeding or random mutagenesis. Accordingly, provided herein are methods and compositions for modifying determinacy in plants that may be used to achieve such benefits, including, for example, development of semi-determinate plants offering unique benefits to growers, despite markets that to date predominately consisted of only determinate and indeterminate varieties in crops such as soybeans.

To produce soybean and other plants having novel growth habit phenotypes, the present disclosure provides, in certain embodiments, methods and compositions for the creation of novel alleles at the Dt1 locus via editing of the TFL1b gene promoter. For example, the promoter region upstream of the TFL1b gene was modified as disclosed herein by co-expressing eight guide RNAs targeting approximately 1.2 kb of the sequence. Edited individuals harboring a series of deletions from 30 to 1746 bp were selected and evaluated. Surprisingly, it was shown that as a result edited dt1 lines could be created representing a series of alleles at the Dt1 locus with an apparent spectrum of determinacy phenotypes, classified as ranging from determinate to super-indeterminate. Alleles that conferred a range of terminal flowering dates extending from 3 weeks before that of the indeterminate background variety to almost a week after were generated, permitting for the first time the ability to engineer the optimal degree of semi-determinacy. Therefore, the present disclosure represents a significant advance in the art in that it permits the production of novel engineered alleles in soybean and other crops that confer novel semi-determinate phenotypes with the potential to protect yield by reducing lodging and thereby increasing per plant productivity.

### I. Genome Editing

The present disclosure provides, in certain embodiments, plants, plant parts, plant cells, and seeds produced through genome modification using site-specific integration or genome editing. Genome editing can be used to make one or more edit(s) or mutation(s) at a desired target site in the genome of a plant, such as to change expression and/or activity of one or more genes, or to integrate an insertion sequence or transgene at a desired location in a plant genome. Any site or locus within the genome of a plant may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE (transcription activator-like effector)-endonuclease (TALEN), a recombinase, or a transposase. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution or insertion, whereas “edits” or “genomic edits” refers to two or more targeted mutation(s), deletion(s), inversion(s), substitution(s) and/or insertion(s), with each “edit” being introduced via a targeted genome editing technique.

According to some embodiments, a site-specific nuclease may be co-delivered with a donor template molecule to serve as a template for making a desired edit, mutation or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease provided herein may be selected from the group consisting of a zinc-finger nuclease (ZFN), a TALE-endonuclease (TALEN), a meganuclease, an RNA-guided endonuclease, a recombinase, a transposase, or any combination thereof. See, e.g., Khandagale et al. (Plant Biotechnol Rep 10:327-343, 2016); and Gaj et al. (Trends Biotechnol. 31(7):397-405, 2013. Zinc finger nucleases (ZFN) are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site but may typically be composed of 3-4 (or more) zinc-fingers. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. Patent App. Pub. Nos. 2005/0064474, 2009/0117617, and 2012/0142062. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al., PLoS One 8:e82539, 2013). MutH is capable of introducing strand-specific nicks in DNA (see Gabsalilow et al., Nucleic Acids Research. 41:e83, 2013). TevI introduces double-stranded breaks in DNA at targeted sites (see Beurdeley et al., Nature Communications 4:1762, 2013).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al. (Nucleic Acids Research 40:W117-122, 2012); Cermak et al. (Nucleic Acids Research 39:e82, 2011); and tale-nt.cac.cornell.edu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions of any thereof, as well as Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 enzyme. The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. The guide RNA may be transformed or introduced into a plant cell or tissue as a gRNA molecule, or as a recombinant DNA molecule, construct or vector comprising a transcribable DNA sequence encoding the guide RNA operably linked to a promoter. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR-associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

As mentioned above, a target gene for genome editing may be any of the TERMINAL FLOWER 1-like genes described herein for suppression, including the soybean TERMINAL FLOWER 1b (GmTFL1b) gene. For knockdown mutations of the TFL1 gene through genome editing, an RNA-guided endonuclease may be targeted to an upstream or downstream sequence, such as a promoter and/or enhancer sequence, or an intron, 5′UTR, and/or 3′UTR sequence of the TFL1 gene to mutate one or more promoter and/or regulatory sequences of the TFL1 gene to affect or reduce its level of expression. For knockdown of the GmTFL1b gene in soybean, a guide RNA may be used, which comprises a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NOs:3 or 4 or a sequence complementary thereto (e.g., 12, 13, 14, 15, 16, 17, 18, 19, 20, 21, 22, 23, 24, 25 or more consecutive nucleotides of SEQ ID NOs:3 or 4 or a sequence complementary thereto), although alternative splicing and different exon/intron boundaries may occur. As used herein, the term “consecutive” in reference to a polynucleotide or protein sequence means without deletions or gaps in the sequence.

As used herein, with respective to a given sequence, a “complement”, a “complementary sequence” and a “reverse complement” are used interchangeably. All three terms refer to the inversely complementary sequence of a nucleotide sequence, i.e. to a sequence complementary to a given sequence in reverse order of the nucleotides.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2(2):59-70, 2014). The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence.

In some embodiments, a site-specific nuclease is a recombinase. Non-limiting examples of recombinases that may be used include a serine recombinase attached to a DNA recognition motif, a tyrosine recombinase attached to a DNA recognition motif, or any recombinase enzyme known in the art attached to a DNA recognition motif. In certain embodiments, the site-specific nuclease is a recombinase or transposase, which may be a DNA transposase or recombinase attached or fused to a DNA binding domain. Non-limiting examples of recombinases include a tyrosine recombinase selected from the group consisting of a Cre recombinase, a Gin recombinase, a Flp recombinase, and a Tnp 1 recombinase attached to a DNA recognition motif provided herein. In one aspect of the present disclosure, a Cre recombinase or a Gin recombinase provided herein is tethered to a zinc-finger DNA-binding domain, a TALE DNA-binding domain, or a Cas9 nuclease. In another aspect, a serine recombinase selected from the group consisting of a PhiC31 integrase, an R4 integrase, and a TP-901 integrase may be attached to a DNA recognition motif provided herein. In yet another aspect, a DNA transposase selected from the group consisting of a TALE-piggyBac and TALE-Mutator may be attached to a DNA binding domain provided herein.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such a genomic locus. The DSB or nick created by the non-RNA-guided site specific nuclease may lead to knockdown of gene expression via repair of the DSB or nick, which may result in a mutation or insertion of a sequence at the site of the DSB or nick through cellular repair mechanisms. Such cellular repair mechanism may be guided by a donor template molecule.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes or transcribable DNA sequences.

Without being limiting, a gene/transgene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

Any method known in the art for suppression of a target gene may be used to suppress a TFL1 gene according to embodiments of the present disclosure, including expression of antisense RNAs, double stranded RNAs (dsRNAs) or inverted repeat RNA sequences, or via co-suppression or RNA interference (RNAi) through expression of small interfering RNAs (siRNAs), short hairpin RNAs (shRNAs), trans-acting siRNAs (ta-siRNAs), or micro RNAs (miRNAs). Furthermore, sense and/or antisense RNA molecules may be used that target the coding and/or non-coding genomic sequences or regions within or near a TFL1 gene to cause silencing of the gene. Accordingly, any of these methods may be used for the targeted suppression of an endogenous TFL1 gene in a tissue-specific or tissue-preferred manner. See, e.g., U.S. Patent Application Publication Nos. 2009/0070898, 2011/0296555, and 2011/0035839.

The introduction of a DSB or nick may also be used to introduce targeted mutations in the genome of a plant. According to this approach, mutations, such as deletions, insertions, inversions, and/or substitutions may be introduced at a target site via imperfect repair of the DSB or nick to produce a knock-out or knock-down of a gene. Such mutations may be generated by imperfect repair of the targeted locus even without the use of a donor template molecule. A “knock-out” of a gene may be achieved by inducing a DSB or nick at or near the endogenous locus of the gene that results in non-expression of the protein or expression of a non-functional protein, whereas a “knock-down” of a gene may be achieved in a similar manner by inducing a DSB or nick at or near the endogenous locus of the gene that is repaired imperfectly at a site that does not affect the coding sequence of the gene in a manner that would eliminate the function of the encoded protein. For example, the site of the DSB or nick within the endogenous locus may be in the upstream or 5′ region of the gene (e.g., a promoter and/or enhancer sequence) to affect or reduce its level of expression.

Similarly, such targeted knock-out or knock-down mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out or knock-down mutations of a gene may be achieved by substituting, inserting, deleting or inverting at least a portion of the gene, such as by introducing a frame shift or premature stop codon into the coding sequence of the gene or disrupting a promoter sequence or the sequence of another non-coding regulatory element of the gene. A deletion of a portion of a gene may also be introduced by generating DSBs or nicks at two target sites and causing a deletion of the intervening target region flanked by the target sites.

In an aspect, the present disclosure provides a modified soybean plant, or plant part thereof, comprising a mutant allele of the TFL1 gene, wherein the mutant allele comprises at least one genome modification involving of at least 10, at least 20, at least 30, at least 40, at least 50, at least 60, at least 70, at least 75, at least 80, at least 85, at least 90, at least 95, at least 100, at least 110, at least 125, at least 150, at least 175, or at least 200 consecutive nucleotides of the promotor region of the endogenous TFL1 gene. The promoter sequence of the soybean TFL1b gene comprises the sequence of SEQ ID NO:4, which is a 2 kb polynucleotide sequence upstream of the transcription initiation site in the TFL1b gene. The genome modification may be a deletion of a region comprising at least 10, at least 20, at least 30, at least 40, at least 50, at least 60, at least 70, at least 75, at least 80, at least 85, at least 90, at least 95, at least 100, at least 110, at least 125, at least 150, at least 175, or at least 200 consecutive nucleotides within the sequence of SEQ ID NO:4. Such a deletion in SEQ ID NO:4 may include a region that spans: from nucleotide 1539 to nucleotide 1568; from nucleotide 1217 to nucleotide 1604; from nucleotide 1518 to nucleotide 1629; from nucleotide 951 to nucleotide 1222; from nucleotide 1364 to nucleotide 1407; from nucleotide 1367 to nucleotide 1516; from nucleotide 754 to nucleotide 1806; from nucleotide 1216 to nucleotide 1319; from nucleotide 1159 to nucleotide 1965; from nucleotide 760 to nucleotide 1214; from nucleotide 939 to nucleotide 1028; from nucleotide 1066 to nucleotide 1664; from nucleotide 952 to nucleotide 1552; from nucleotide 1677 to nucleotide 1808; from nucleotide 1524 to nucleotide 1558; from nucleotide 476 to nucleotide 1405; from nucleotide 1365 to nucleotide 1559; from nucleotide 928 to nucleotide 1560; or from nucleotide 593 to nucleotide 1813 of SEQ ID NO:4.

In an aspect, the genome modification may be an inversion of a region of at least 10, at least 20, at least 30, at least 40, at least 50, at least 60, at least 70, at least 75, at least 80, at least 85, at least 90, at least 95, at least 100, at least 110, at least 125, at least 150, at least 175, or at least 200 consecutive nucleotides within the sequence of SEQ ID NO:4. Such an inversion may comprise a region from nucleotide 1029 to nucleotide 1065 of SEQ ID NO:4 that has been removed, inverted, and reinserted at the same location in SEQ ID NO:4. In an aspect, a mutant allele of the TFL1 gene may comprise two or more modifications in the promotor region of the endogenous TFL1 gene. Examples of such mutant alleles of the soybean TFL1b gene are disclosed herein and include, for example, an allele comprising two deletions in the sequence of SEQ ID NO:4, wherein the first deletion spans a region from nucleotide 952 to nucleotide 1552 of SEQ ID NO:4 and the second deletion spans from nucleotide 1677 to nucleotide 1808 of SEQ ID NO:4; and an allele comprising two deletions and an inversion within the sequence of SEQ ID NO:4, where the first deletion spans a region from nucleotide 939 to nucleotide 1028 of SEQ ID NO:4 and the second deletion spans from nucleotide 1066 to nucleotide 1664 of SEQ ID NO:4, and the inversion comprises a region spanning from nucleotide 1029 to nucleotide 1065 of SEQ ID NO:4 that has been removed, inverted, and reinserted at the same location in SEQ ID NO:4.

Other targeted modifications may be made in the promotor region to generate novel alleles in the soybean TFL1b gene and homologs thereof. SEQ ID NOs:67-77, 79, and 81 each represent an approximately 2 kb polynucleotide sequence upstream of the transcription initiation site in the TFL1b gene homologs found in Zea mays (SEQ ID NO:67), Sorghum bicolor (SEQ ID NO:68), Oryza sativa (SEQ ID NO:69), Triticum aestivum (SEQ ID NO:70), Hordeum vulgare (SEQ ID NO:71), Solanum lycopersicum (SEQ ID NO:72), Gossypium hirsutum (SEQ ID NO:73), Capsicum annuum (SEQ ID NO:74), Brassica napus (SEQ ID NO:75), Arabidopsis thaliana (SEQ ID NO:76), Medicago truncatula (SEQ ID NO:77), Arachis hypogaea (SEQ ID NO:79), and Cicer arietinum (SEQ ID NO:81). For example, one or more modification sites may be located at about 200 nucleotides from or greater than 200 nucleotides from the 3′ end of the sequences of SEQ ID NOs:4, 67-77, 79, and 81. In an aspect, one or more modifications may be made within the region of DNA spanning from nucleotide position 1237 to nucleotide position 1570 of SEQ ID NO:4 to generate a novel allele in the soybean TFL1b gene.

In a further aspect, the present disclosure provides a modified soybean plant, or plant part thereof, comprising a mutant allele of the TFL1 gene, wherein the mutant allele comprises one or more junction sequences, wherein the junction sequences are at least 30, at least 60, at least 100 nucleotides at the junction site. As used herein a “junction” or “junction site” is the connection point between the nucleotide sequences at the site of a deletion, insertion, substitution, or inversion. In the case of a deletion, the junction is the connection point at the site of the deletion of the sequences that previously flanked the deletion. For example, in the case of the 30 base pair deletion from nucleotide 1539 to nucleotide 1568, as compared to reference sequence SEQ ID NO:4 described herein, the junction would be between nucleotide 1538 and nucleotide 1569. In the case of an insertion, substitution, or inversion, the junction is the connection point between the inserted, inverted, or substituted sequence and the flanking DNA sequences. In the case of an insertion, substitution, or inversion, one junction is found at the 5′ end of the insertion, substitution or inversion, and another junction is found at the 3′ end of the insertion, substitution, or inversion. A “junction sequence” refers to a DNA sequence of any length that spans a junction. A junction sequence can comprise at least 10 nucleotides, at least, 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, or more.

### II. Constructs for Genome Editing

Recombinant DNA constructs and vectors are provided comprising a polynucleotide sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA constructs and vectors are further provided comprising a polynucleotide sequence encoding a guide RNA, wherein the guide RNA comprises a guide sequence of sufficient length having a percent identity or complementarity to a target site within the genome of a plant, such as at or near a targeted TFL1 gene. A polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have a reduced or eliminated activity or expression level for the gene relative to the wild-type allele. For example, a mutant or edited allele for TFL1 gene may have a deletion in the promoter region upstream of the endogenous TFL1 gene. For diploid organisms such as soybean, a first allele can occur on one chromosome, and a second allele can occur at the same locus on a second homologous chromosome. If one allele at a locus on one chromosome of a plant is a mutant or edited allele and the other corresponding allele on the homologous chromosome of the plant is wild-type, then the plant is described as being heterozygous for the mutant or edited allele. However, if both alleles at a locus are mutant or edited alleles, then the plant is described as being homozygous for the mutant or edited alleles. A plant homozygous for mutant or edited alleles at a locus may comprise the same mutant or edited allele or different mutant or edited alleles if heteroallelic or biallelic.

As used herein, a “wild-type gene” or “wild-type allele” refers to a gene or allele having a sequence or genotype that is most common in a particular plant species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions or any desired combinations of such changes in a native polynucleotide or amino acid sequence.

As used herein, the term “expression” refers to the biosynthesis of a gene product, and typically the transcription and/or translation of a nucleotide sequence, such as an endogenous gene, a heterologous gene, a transgene or an RNA and/or protein coding sequence, in a cell, tissue, organ, or organism, such as a plant, plant part or plant cell, tissue or organ.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc. The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

Reference in this application to an “isolated DNA molecule” or an “isolated polynucleotide”, or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding an protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. A promoter provided herein, or variant or fragment thereof, may comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction—e.g., the promoter or regulatory sequence has a different origin relative to the associated polynucleotide sequence and/or the promoter or regulatory sequence is not naturally occurring in a plant species to be transformed with the promoter or regulatory sequence.

As used herein, an “endogenous gene” or an “endogenous locus” refers to a gene or locus at its natural and original chromosomal location. As used herein, the “endogenous TFL1 gene” refers to the TFL1 genic locus at its original chromosomal location.

As used herein, in the context of a protein-coding gene, an “exon” refers to a segment of a DNA or RNA molecule containing information coding for a protein or polypeptide sequence.

As used herein, an “intron” of a gene refers to a segment of a DNA or RNA molecule, which does not contain information coding for a protein or polypeptide, and which is first transcribed into an RNA sequence but then spliced out from a mature RNA molecule.

As used herein, an “untranslated region (UTR)” of a gene refers to a segment of an RNA molecule or sequence (e.g., a mRNA molecule) expressed from a gene (or transgene), but excluding the exon and intron sequences of the RNA molecule. An “untranslated region (UTR)” also refers a DNA segment or sequence encoding such a UTR segment of an RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “transcription termination sequence” refers to a nucleic acid sequence containing a signal that triggers the release of a newly synthesized transcript RNA molecule from an RNA polymerase complex and marks the end of transcription of a gene or locus.

As used herein, a “homolog” or “homologues” means a protein in a group of proteins that perform the same biological function, for example, proteins that belong to the same TFL1-like protein family and that provide a common enhanced trait in modified plants of this disclosure. Homologs are expressed by homologous genes. With reference to homologous genes, homologs include orthologs, for example, genes expressed in different species that evolved from common ancestral genes by speciation and encode proteins retain the same function, but do not include paralogs, i.e., genes that are related by duplication but have evolved to encode proteins with different functions. Homologous genes include naturally occurring alleles and artificially-created variants.

The terms “percent identity,” “% identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 70%, at least about 75%, at least about 80%, at least about 85%, at least about 90%, at least about 92%, at least about 93%, at least about 94%, at least about 95%, at least about 96%, at least about 97%, at least about 98%, at least about 99%, or even at least about 99.5% identity over the full length of a protein or its corresponding nucleotide sequence identified as being associated with imparting an altered determinacy phenotype when expressed in plant cells. According to embodiments of the present invention, a TFL1 gene or homolog thereof encodes a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2. Examples of homologs of the soybean TFL1b protein (SEQ ID NO:2) include, but are not limited to, the sequences of SEQ ID NOs:52-66, 78, and 80.

Homologs are inferred from sequence similarity, by comparison of protein sequences, for example, manually or by use of a computer-based tool. For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST, can also be used, for example to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Methods for producing such fragments from a starting promoter molecule are well known in the art. Fragments of a DNA molecule or protein may exhibit the activity of the DNA molecule or protein from which they are derived.

According to another aspect, the present disclosure provides methods for altering a phenotype, such as increasing yield, altering determinacy, or reducing lodging in a plant comprising: (a) modifying the genome of a plant cell by: (i) identifying an endogenous gene of the plant corresponding to the a soybean TFL1 gene, such as GmTFL1b gene described herein, and its homologs, and (ii) modifying the promotor sequence of the endogenous gene in the plant cell via targeted mutagenesis to modify the expression level of the endogenous gene; and (b) regenerating or developing a plant from the plant cell. Various TFL1 genes and proteins from different plant species may be identified and considered TFL1 homologs or orthologs for use in the present disclosure if they have a similar nucleic acid and/or protein sequence and share conserved amino acids and/or structural domain(s) with at least one known TFL1 gene or protein.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing or regenerating transformed explants, tissues, plants and/or plant parts.

### III. Transformation Methods

Methods and compositions are provided for transforming a plant cell, tissue or explant with a recombinant DNA molecule or construct encoding one or more molecules required for targeted genome editing (e.g., guide RNA(s) and/or site-directed nuclease(s)). Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially-mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616. Other methods for plant transformation, such as microinjection, electroporation, vacuum infiltration, pressure, sonication, silicon carbide fiber agitation, PEG-mediated transformation, etc., are also known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants. Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating” refer to a process of growing or developing a plant from one or more plant cells through one or more culturing steps. Transformed or edited cells, tissues or explants containing a DNA sequence insertion or edit may be grown, developed or regenerated into transgenic plants in culture, plugs, or soil according to methods known in the art. Certain embodiments of the disclosure therefore relate to methods and constructs for regenerating a plant from a cell with modified genomic DNA resulting from genome editing. The regenerated plant can then be used to propagate additional plants.

According to an aspect of the present disclosure, regenerated plants or a progeny plant, plant part or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait or phenotype is recessive, one or more generations or crosses (e.g., selfing) from the initial R₀ plant may be necessary to produce a plant homozygous for the edit or mutation so the trait or phenotype can be observed. Progeny plants, such as plants grown from R₁ seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot; PCR; enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

A plant that may be transformed with a recombinant DNA molecule or transformation vector comprising a guide RNA may include a variety of flowering plants or angiosperms, which may be further defined as including various dicotyledonous (dicot) plant species or monocotyledonous (monocot) plant species. A dicot plant could be members of the Fabaceae family (such as legumes), sunflower (Helianthus annuus), safflower (Carthamus tinctorius), sesame (Sesamum spp.), tobacco (Nicotiana tabacum), potato (Solanum tuberosum), cotton (Gossypium barbadense, Gossypium hirsutum), sweet potato (Ipomoea batatas), cassava (Manihot esculenta), coffee (Coffea spp.), tea (Camellia spp.), fruit trees, such as apple (Malus spp.), Prunus spp., such as plum, apricot, peach, cherry, etc., pear (Pyrus spp.), fig (Ficus carica), etc., citrus trees (Citrus spp.), cocoa (Theobroma cacao), avocado (Persea americana), olive (Olea europaea), almond (Prunus amygdalus), walnut (Juglans spp.), strawberry (Fragaria spp.), watermelon (Citrullus lanatus), pepper (Capsicum spp.), beet (Beta vulgaris), grape (Vitis, Muscadinia), tomato (Lycopersicon esculentum, Solanum lycopersicum), cucumber (Cucumis sativus), and members of the Brassicaceae family, such as thale cress (Arabidopsis thaliana) and Brassica sp. (e.g., B. napus, B. rapa, B. juncea), particularly those Brassica species useful as sources of seed oil. Legumes and leguminous plants include peas (Pisum sativum) alfalfa (Medicago sativa), barrel clover (Medicago truncatula), pigeon pea (Cajanus cajan) guar (Cyamopsis tetragonoloba), carob (Ceratonia siliqua), fenugreek (Trigonella foenum-graecum), soybean (Glycine max), common bean (Phaseolus vulgaris), cowpea (Vigna unguiculata), mung bean (Vigna radiata), lima bean (Phaseolus lunatus), fava bean (Vicia faba), lentil (Lens culinaris or Lens esculenta), peanut (Arachis hypogaea), licorice (Glycyrrhiza glabra), and chickpea (Cicer arietinum). A monocot plant could be oil palm (Elaeis spp.), coconut (Cocos spp.), banana (Musa spp.), and cereals such as corn (Zea mays), barley (Hordeum vulgare), sorghum (Sorghum bicolor), rice (Oryza sativa), and wheat (Triticum aestivum). Given that the present disclosure may apply to a broad range of plant species, the present disclosure further applies to other botanical structures analogous to pods of leguminous plants, such as bolls, siliques, fruits, nuts, tubers, etc.

### IV. Genome Modified Plants

As used herein, “modified” in the context of a plant, plant seed, plant part, plant cell, and/or plant genome, refers to a plant, plant seed, plant part, plant cell, and/or plant genome comprising an engineered change in the expression level and/or endogenous sequence of one or more genes of interest relative to a wild-type or control plant, plant seed, plant part, plant cell, and/or plant genome. Indeed, the term “modified” may further refer to a plant, plant seed, plant part, plant cell, and/or plant genome having one or more deletions affecting expression of an endogenous TFL1 gene introduced through chemical mutagenesis, transposon insertion or excision, or any other known mutagenesis technique, or introduced through genome editing. In an aspect, a modified plant, plant seed, plant part, plant cell, and/or plant genome can comprise one or more transgenes. For clarity, therefore, a modified plant, plant seed, plant part, plant cell, and/or plant genome includes a mutated, edited and/or transgenic plant, plant seed, plant part, plant cell, and/or plant genome having a modified expression level, expression pattern, and/or sequence of a TFL1 gene relative to a wild-type or control plant, plant seed, plant part, plant cell, and/or plant genome.

Modified plants, plant parts, seeds, etc., may have been subjected to mutagenesis, genome editing or site-directed integration, genetic transformation, or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the TFL1 gene. A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the DNA sequence or construct or edit. For example, a DNA sequence or inversion may be introduced into a first plant line that is amenable to transformation or editing, which may then be crossed with a second plant line to introgress the DNA sequence or edit (e.g., deletion) into the second plant line. Progeny of these crosses can be further backcrossed into the desirable line multiple times, such as through 6 to 8 generations or back crosses, to produce a progeny plant with substantially the same genotype as the original parental line, but for the introduction of the DNA sequence or edit. A modified plant, plant cell, or seed provided herein may be a hybrid plant, plant cell, or seed. As used herein, a “hybrid” is created by crossing two plants from different varieties, lines, inbreds, or species, such that the progeny comprises genetic material from each parent. Skilled artisans recognize that higher order hybrids can be generated as well.

A modified plant, plant part, plant cell, or seed provided herein may be of an elite variety or an elite line. An “elite variety” or an “elite line” refers to a variety that has resulted from breeding and selection for superior agronomic performance.

As used herein, the term “control plant” (or likewise a “control” plant seed, plant part, plant cell, and/or plant genome) refers to a plant (or plant seed, plant part, plant cell, and/or plant genome) that is used for comparison to a modified plant (or modified plant seed, plant part, plant cell, and/or plant genome) and has the same or similar genetic background (e.g., same parental lines, hybrid cross, inbred line, testers, etc.) as the modified plant (or plant seed, plant part, plant cell, and/or plant genome), except for genome edit(s) (e.g., a deletion) affecting a TFL1 gene. For example, a control plant may be an inbred line that is the same as the inbred line used to make the modified plant, or a control plant may be the product of the same hybrid cross of inbred parental lines as the modified plant, except for the absence in the control plant of any transgenic events or genome edit(s) affecting a TFL1 gene. Similarly, an “unmodified control plant” refers to a plant that shares a substantially similar or essentially identical genetic background as a modified plant, but without the one or more engineered changes to the genome (e.g., mutation or edit) of the modified plant. For purposes of comparison to a modified plant, plant seed, plant part, plant cell, and/or plant genome, a “wild-type plant” (or likewise a “wild-type” plant seed, plant part, plant cell, and/or plant genome) refers to a non-transgenic and non-genome edited control plant, plant seed, plant part, plant cell, and/or plant genome. As used herein, a “control” plant, plant seed, plant part, plant cell, and/or plant genome may also be a plant, plant seed, plant part, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the terms “suppress,” “suppression,” “inhibit,” “inhibition,” “inhibiting,” “knockout,” “knockdown,” and “downregulation” refer to a lowering, reduction, or elimination of the expression level of an mRNA and/or protein encoded by a target gene in a plant, plant cell, or plant tissue at one or more stage(s) of plant development, as compared to the expression level of such target mRNA and/or protein in a wild-type or control plant, cell, or tissue at the same stage(s) of plant development. According to some embodiments, a modified plant is provided having a TFL1 gene expression level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to further embodiments, a modified plant is provided having a TFL1 gene expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

According to some embodiments, a modified plant is provided having a TFL1 mRNA level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a TFL1 mRNA expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a modified plant is provided having a TFL1 protein expression level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a TFL1 protein expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

The present disclosure relates to a plant with improved economically important characteristics, including but not limited to increased yield, increased determinacy, reduced time to reach terminal flowering date, reduced lodging rate, and reduced susceptibility to fungal disease. More specifically with respect to yield, the present disclosure relates to a modified plant comprising a genomic edit or mutation as described herein, wherein the plant has increased yield as compared to a control plant. Many plants of this disclosure exhibited increased yield or improved yield trait components as compared to a control plant. Yield can be defined as the measurable produce of economic value from a crop. Yield can be defined in the scope of quantity and/or quality. For example, soybean yield can include pods per plant, pods per acre, seeds per plant, seeds per pod, weight per seed, weight per pod, pods per node, number of nodes, and the number of internodes per plant. Yield can be directly dependent on several factors, for example, the number and size of organs, plant architecture (such as the number of branches, plant biomass, etc.), flowering time and duration, grain fill period. Root architecture and development, photosynthetic efficiency, nutrient uptake, stress tolerance, early vigor, delayed senescence and functional stay green phenotypes can be important factors in determining yield. Optimizing the above-mentioned factors can therefore contribute to increasing crop yield.

Modified plants comprising or derived from plant cells that are transformed with a recombinant DNA of this disclosure can be further enhanced with stacked traits, for example, a modified crop plant having an enhanced trait resulting from expression of DNA disclosed herein in combination with one or more genes of agronomic interest that provide a beneficial agronomic trait (such as herbicide and/or pest resistance traits) to crop plants. For example, the traits conferred by the recombinant DNA constructs of the current disclosure can be stacked with other traits of agronomic interest, such as a trait providing insect resistance such as using a gene from Bacillus thuringensis to provide resistance against lepidopteran, coleopteran, homopteran, hemiopteran, and other insects, or improved quality traits such as improved nutritional value. Molecules and methods for imparting insect/nematode/virus resistance are disclosed in U.S. Pat. Nos. 5,250,515; 5,880,275; 6,506,599; 5,986,175; and U.S. Patent Application Publication No.

Herbicides for which transgenic plant tolerance has been demonstrated and the methods and compositions of the present disclosure can be applied include, but are not limited to, glyphosate, dicamba, glufosinate, sulfonylurea, bromoxynil, norflurazon, 2,4-D (2,4-dichlorophenoxy) acetic acid, aryloxyphenoxy propionates, p-hydroxyphenyl pyruvate dioxygenase inhibitors (HPPD), and protoporphyrinogen oxidase inhibitors (PPO) herbicides. Polynucleotide molecules encoding proteins involved in herbicide tolerance known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent No. Application Publication No. 2003/0083480 A1 also for imparting glyphosate tolerance; dicamba monooxygenase disclosed in U.S. Patent Application Publication No. 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al. (Plant J. 4:833-840, 1993) and in Misawa et al. (Plant J. 6:481-489, 1994) for norflurazon tolerance; a polynucleotide molecule encoding acetohydroxyacid synthase (AHAS, aka ALS) described in Sathasivan et al. (Nucl. Acids Res. 18:2188-2193, 1990) for imparting tolerance to sulfonylurea herbicides; polynucleotide molecules known as bar genes disclosed in DeBlock et al. (EMBO J. 6:2513-2519, 1987) for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260.

Genetic elements, methods, and transgenes that confer fungal disease resistance may also be used with the present disclosure (U.S. Pat. Nos. 6,653,280; 6,573,361; 6,506,962; 6,316,407; 6,215,048; 5,516,671; 5,773,696; 6,121,436; 6,316,407; 6,506,962). Soybean diseases caused by fungi include, but are not limited to, Phakopsora pachyrhizi, Phakopsora meibomiae (Asian Soybean Rust), Colletotrichum truncatum, Colletotrichum dematium var. truncatum, Glomerella glycines (Soybean Anthracnose), Phytophthora sojae (Phytophthora root and stem rot), Sclerotinia sclerotiorum (Sclerotinia stem rot), Fusarium solani f. sp. glycines (sudden death syndrome), Fusarium spp. (Fusarium root rot), Macrophomina phaseolina (charcoal rot), Septoria glycines, (Brown Spot), Pythium aphanidermatum, Pythium debaryanum, Pythium irregulare, Pythium ultimum, Pythium myriotylum, Pythium torulosum (Pythium seed decay), Diaporthe phaseolorum var. sojae (Pod blight), Phomopsis longicola (Stem blight), Phomopsis spp. (Phomopsis seed decay), Peronospora manshurica (Downy Mildew), Rhizoctonia solani (Rhizoctonia root and stem rot, Rhizoctonia aerial blight), Phialophora gregata (Brown Stem Rot), Diaporthe phaseolorum var. caulivora (Stem Canker), Cercospora kikuchii (Purple Seed Stain), Alternaria sp. (Target Spot), Cercospora sojina (Frogeye Leafspot), Sclerotium rolfsii (Southern blight), Arkoola nigra (Black leaf blight), Thielaviopsis basicola, (Black root rot), Choanephora infundibulifera, Choanephora trispora (Choanephora leaf blight), Leptosphaerulina trifolii (Leptosphaerulina leaf spot), Mycoleptodiscus terrestris (Mycoleptodiscus root rot), Neocosmospora vasinfecta (Neocosmospora stem rot), Phyllosticta sojicola (Phyllosticta leaf spot), Pyrenochaeta glycines (Pyrenochaeta leaf spot), Cylindrocladium crotalariae (Red crown rot), Dactuliochaeta glycines (Red leaf blotch), Spaceloma glycines (Scab), Stemphylium botryosum (Stemphylium leaf blight), Corynespora cassiicola (Target spot), Nematospora coryli (Yeast spot), and Phymatotrichum omnivorum (Cotton Root Rot).

### V. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and plant transformation.

When introducing elements of the present disclosure or the embodiment(s) thereof, the articles “a”, “an”, “the”, and “said” are intended to mean that there are one or more of the elements.

The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “plant” includes a whole plant, explant, plant part, seedling, or plantlet at any stage of regeneration or development.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

An “embryo” is a part of a plant seed, consisting of precursor tissues (e.g., meristematic tissue) that can develop into all or part of an adult plant. An “embryo” may further include a portion of a plant embryo.

A “meristem” or “meristematic tissue” comprises undifferentiated cells or meristematic cells, which are able to differentiate to produce one or more types of plant parts, tissues or structures, such as all or part of a shoot, stem, root, leaf, seed, etc.

As used herein, “determinate growth habit” refers to a cease vegetative growth after the main stem terminates in a cluster of mature pods. Determinate soybean varieties begin flowering when all or most of the nodes on the main stem have developed. They usually have elongated racemes that may be several centimeters in length and may have a large number of flowers. As used herein, “indeterminate growth habit” refers to the development of leaves and flowers simultaneously throughout a portion of their reproductive period, with one to three pods at the terminal apex. Indeterminate soybean varieties, when grown at their latitude of adaptation, flower when about one-half of the nodes on the main stem have developed. They have short racemes with few flowers, and their terminal node has only a few flowers. “Semi-determinate” soybean varieties also flower when about one-half of the nodes on the main stem have developed, but node development and flowering on the main stem stops more abruptly than on indeterminate varieties. Their racemes are short and have few flowers, except for the terminal one, which may have several times more flowers than those lower on the plant.

As used herein “lodging” refers to the bending over of the stems near ground level in plants of grain crops. Lodging is rated on a scale of 1 to 9. Generally, a score of 1 indicates erect plants. A score of 5 indicates plants are leaning at a 45 degree(s) angle in relation to the ground and a score of 9 indicates plants are laying on the ground. Table 4 shows the lodging scale used herein to evaluate control and modified soybean plants.

As used herein “terminal flowering date” refers the date on which there is one open flower at the terminal node of the main stem on 50% of the plants in a uniform plot segment.

As used herein, the “vegetative phase” of plant development is the period of growth between germination and flowering. The stages in the vegetative phase of soybean are as follows: VE (emergence), VC (cotyledon stage), V1 (first trifoliolate leaf), V2 (second trifoliolate leaf), V3 (third trifoliolate leaf), V(n) (nth trifoliolate leaf), and V6 (flowering will soon start). As used herein, the “reproductive phase” of plant development is the period between flowering and the end of harvest. The stages in the reproductive phase of soybean are as follows R1 (beginning bloom, first flower); R2 (full bloom, flower in top 2 nodes); R3 (beginning pod, 3/16″ pod in top 4 nodes); R4 (full pod, ¾″ pod in top 4 nodes); R5 (⅛″ seed in top 4 nodes); R6 (full size seed in top 4 nodes); R7 (beginning maturity, one mature pod); and, R8 (full maturity, 95% of pods on the plant have reached mature color). Soybean vegetative and reproductive stages are well known to those of skill in the art and numerous publications describing these stages can be found on the world wide web and elsewhere, such as North Dakota State University publication A-1174, June 1999, Reviewed and Reprinted August 2004.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

Having described the present disclosure in detail, it will be apparent that modifications, variations, and equivalent embodiments are possible without departing from the spirit and scope of the present disclosure as further defined in the appended claims. Furthermore, it should be appreciated that all examples in the present disclosure including the following are provided as non-limiting examples.

### EXAMPLES

### Example 1. Design of Gene Editing Constructs

Employing gene editing to perform targeted mutagenesis of determinacy loci offers the opportunity to create a alleles that confer a range of determinacy phenotypes, which in turn, results in diversity in soybean plant architecture and growth habit. Specific endogenous expression of the dominant allele of the soybean Dt1 locus, the GmTFL1b gene, at stem tips protects the apical meristem from terminal differentiation, leading to indeterminacy. The level of determinacy can be modulated by varying the expression level of the GmTFL1b gene through modification of the promoter region of GmTFL1b gene. The coding sequence of the GmTFL1b gene is provided as SEQ ID NO:1, the amino acid sequence for the TFL1b protein is provided as SEQ ID NO:2, and the cDNA sequence for the GmTFL1b gene, including 5′ UTR and 3′UTR, is provided as SEQ ID NO:3.

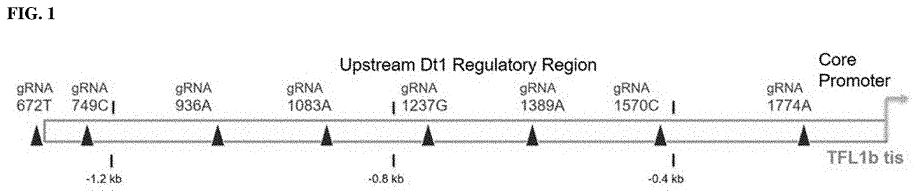

The upstream promoter region of the GmTFL1b gene was targeted for mutagenesis using gene editing. Four gene editing constructs for plant transformation were designed with multiple guide RNAs (gRNAs) to target various locations within a 2 kb promoter region (SEQ ID NO:4) upstream of the transcription initiation site (tis) of the GmTFL1b gene, as illustrated in FIG. 1.

Each of the plant transformation vector constructs was designed to make double-stranded breaks (DSBs) at multiple locations as targeted by the gRNAs. Small deletions at DSB sites are possible, as are deletions of large segments between DSB sites.

In this example, the genome editing constructs generally each contained two to three functional regions or cassettes relevant to gene editing and creation of the DSBs in the GmTFL1b gene promoter region: expression of a Cpf1 protein, expression of two to four gRNAs targeting the GmTFL1b gene promoter region and, optionally, expression of an additional four gRNAs targeting the GmTFL1b gene promoter region. Each gRNA unit contains a common scaffold compatible with the Cpf1 gene (SEQ ID NO:5), and a unique spacer/targeting sequence complementary to its intended target site as listed in Table 1. The DNA sequences encoding the gRNA spacers and their intended target sites are listed in Table 1.

TABLE 1
Example guide RNAs used for editing the promoter region of the endogenous
GmTFL1b gene.

| | guide RNA | | | SEQ ID |
|---|---|---|---|---|
| Construct | spacer | Target Site | Spacer Sequence | NO |
| pM552 | g + 1774A | pos. 1774 to pos. 1796 | TGACCAGCGGAGAGAGAAAGAGA | 13 |
| | g − 1570C | pos. 1548 to pos. 1570 | TCTCCCATCAGCTCGATCAGGTC | 14 |
| | g − 1389A | pos. 1367 to pos. 1389 | GTATACTACTAGTACCATGGACG | 15 |
| | g − 1237G | pos. 1215 to pos. 1237 | TTGGAAGATTCCTGATGAAGAGG | 16 |
| | g + 1083A | pos. 1083 to pos. 1105 | CCAATATTGGAACTTGGTCTGCA | 17 |
| | g + 936A | pos. 936 to pos. 958 | AGCACGGGATGTGCATGTACACA | 18 |
| | g + 749C | pos. 749 to pos. 771 | ACAGTGAAACGATACCAACCTGC | 19 |
| | g − 672T | pos. 650 to pos. 672 | TTGGTCTGAAACTGTTAGTTGCT | 20 |
| pM206 | g − 1570C | pos. 1548 to pos. 1570 | TCTCCCATCAGCTCGATCAGGTC | 21 |
| | g − 1389A | pos. 1367 to pos. 1389 | GTATACTACTAGTACCATGGACG | 22 |
| | g − 1237G | pos. 1215 to pos. 1237 | TTGGAAGATTCCTGATGAAGAGG | 23 |
| pM205 | g − 1570C | pos. 1548 to pos. 1570 | TCTCCCATCAGCTCGATCAGGTC | 24 |
| | g − 1237G | pos. 1215 to pos. 1237 | TTGGAAGATTCCTGATGAAGAGG | 25 |
| pM207 | g − 1389A | pos. 1367 to pos. 1389 | GTATACTACTAGTACCATGGACG | 26 |
| | g − 1237G | pos. 1215 to pos. 1237 | TTGGAAGATTCCTGATGAAGAGG | 27 |
| pM204 | g − 1570C | pos. 1548 to pos. 1570 | TCTCCCATCAGCTCGATCAGGTC | 28 |
| | g − 1389A | pos. 1367 to pos. 1389 | GTATACTACTAGTACCATGGACG | 29 |

The Cpf1 expression cassette of editing construct pM552 comprised a Dahlia mosaic virus FLT promoter (SEQ ID NO:6) operably linked to a sequence encoding a Lachnospiraceae bacterium Cpf1 RNA-guided endonuclease enzyme (SEQ ID NO:7) that was codon-optimized for rice, flanked on each side by one copy of a nuclear localization signal (SEQ ID NO:8). See, e.g., Gao et al., (Nature Biotechnol. 35(8):789-792, 2017). The Cpf1 expression cassette in the other four constructs shown in Table 1 comprised a Medicago truncatula ubiquitin promoter (SEQ ID NO:9) operably linked to a sequence codon-optimized for corn encoding a Lachnospiraceae bacterium Cpf1 RNA-guided endonuclease enzyme (SEQ ID NO:10) flanked on each side by one copy of a nuclear localization signal (SEQ ID NO:8).

One type of gRNA expression cassette, present in all of the constructs, comprised a sequence encoding two to four gRNAs operably linked to a soybean RNA polymerase III (Pol3) promoter (SEQ ID NO:11). Spacer sequences as listed in Table 1 targeted alternative breakage sites in the promoter region of GmTFL1b. One additional type of gRNA expression cassette, which is present only in pM552, comprised a sequence encoding another four guide RNAs operably linked to a soy 7SL_CR10 promoter (SEQ ID NO:12). Spacer sequences as listed in Table 1 target alternative breakage sites in promoter region of GmTFL1b.

Specifically, gene editing constructs for plant transformation will be designed to comprise gRNAs targeting a region of DNA spanning from nucleotide position 1237 to nucleotide position 1570 of reference sequence SEQ ID NO:4.

### Example 2. Confirmation of Edits in Promoter Region of GmTFL1b and Zygosity of Genome-Edited Plants

An inbred wild-type soybean line was transformed via Agrobacterium-mediated transformation with the pM552 vector described in Example 1 above. The transformed plant tissue was grown to produce mature R₀ plants. R₀ plants having one or more unique genome edits were self-crossed to produce R₁ plants. R₁ plants that were homozygous for alleles comprising edited GmTFL1b promoter sequences and lacking of editing T-DNA sequences were self-crossed to produce R₂ plants.

To determine the edits made in the GmTFL1b promoter region, an amplicon sequencing technique was used to produce mutant sequences for the 2 kb promoter region for comparison with the wild-type sequence. Amplicon sequencing involves the generation of one or more unique PCR products across the genomic region of interest for next-generation sequencing. Sequence data from each sample is then mapped to a reference sequence to identify differences in the consensus sequences. Plants with unique deletions ranging from 30 to 1221 base pairs (bp) in length were selected to provide diverse coverage of the mutations in the targeted promoter region. Individual R₁ plants produced by selfing R₀ plants having one or more of the edits were assayed for the nature of the edits and the zygosity of the edited mutant or allele and are described in Table 2. All edited plants described in Table 2 were produced using the transformation vector of the pM552 construct. In Table 2, “R₀ Event” is the R₀ plant identifier; “Allele Name” is the identifier for a unique allele; and Null (WT) corresponds to the unedited inbred soybean plant. “Causal Lesion” indicates the starting and ending coordinates of a deleted segment of the promoter, with the 5′ end of the 2 kb promoter sequence (SEQ ID NO:4) as the starting point. “R₁ zygosity” indicates the zygosity of the R₁ plant, as homozygous, heterozygous, not determined (ND), or lethal (for an edit that resulted in a lethal phenotype). Allele AL430a contains three modifications to the endogenous GmTFL1b gene: two deletions (a segment starting at position 939 and ending at position 1028 of SEQ ID NO:4; and a segment starting at position 1066 and ending at position 1664 of SEQ ID NO:4) and an inversion mutation (a segment starting at position 1065 and ending at position 1029 of SEQ ID NO:4 inverted and reinserted into the deleted region).

TABLE 2
Edited plants produced by pM552 editing construct,
with segmental deletion of GmTFL1b promoter.

| | | Causal Deletion | | |
|---|---|---|---|---|
| | | (start and end | | |
| R₀ | | nucleotide positions | R₁ | SEQ |
| Event | Allele Name | of SEQ ID NO: 4) | Zygosity | ID NO |
| S376 | Null (WT) | N/A | no edits | N/A |
| S376 | AL376a | pos. 1539 to pos. 1568 | homozygous | 30 |
| S376 | AL376b | pos. 1217 to pos. 1604 | homozygous | 31 |
| S391 | AL391a | pos. 1518 to pos. 1629 | homozygous | 32 |
| S391 | AL391b | pos. 951 to pos. 1222 | homozygous | 33 |
| S398 | AL398 | pos. 1364 to pos. 1407 | homozygous | 34 |
| S410 | AL410a | pos. 1367 to pos. 1516 | ND | 35 |
| S411 | AL411a | pos. 754 to pos. 1806 | homozygous | 36 |
| S411 | AL411b | pos. 1216 to pos. 1319 | homozygous | 37 |
| S411 | AL411c | pos. 1159 to pos. 1965 | heterozygous | 38 |
| S419 | AL419 | pos. 760 to pos. 1214 | ND | 39 |
| S430 | AL430a | pos. 939 to pos. 1028; | homozygous | 40 |
| | | INV-pos. 1065 to pos. 1029; | | |
| | | pos. 1066 to pos. 1664 | | |
| S430 | AL430b | pos. 952 to pos. 1552; | homozygous | 41 |
| | | pos. 1677 to pos. 1808 | | |
| S437 | AL437 | pos. 1524 to pos. 1558 | homozygous | 42 |
| S447 | AL447a | pos. 476 to pos. 1405 | homozygous | 43 |
| S447 | AL447b | pos. 1365 to pos. 1559 | homozygous | 44 |
| S455 | AL455 | pos. 928 to pos. 1560 | homozygous | 45 |
| S461 | AL461 | pos. 346 to pos. 2092 | lethal | N/A |
| S466 | AL466 | pos. 593 to pos. 1813 | homozygous | 46 |

* The causal lesion listed for each allele represents the largest deletion in the allele, which is believed to lead to the altered determinacy phenotype observed in the modified plants. For some alleles, additional small deletions were also present.

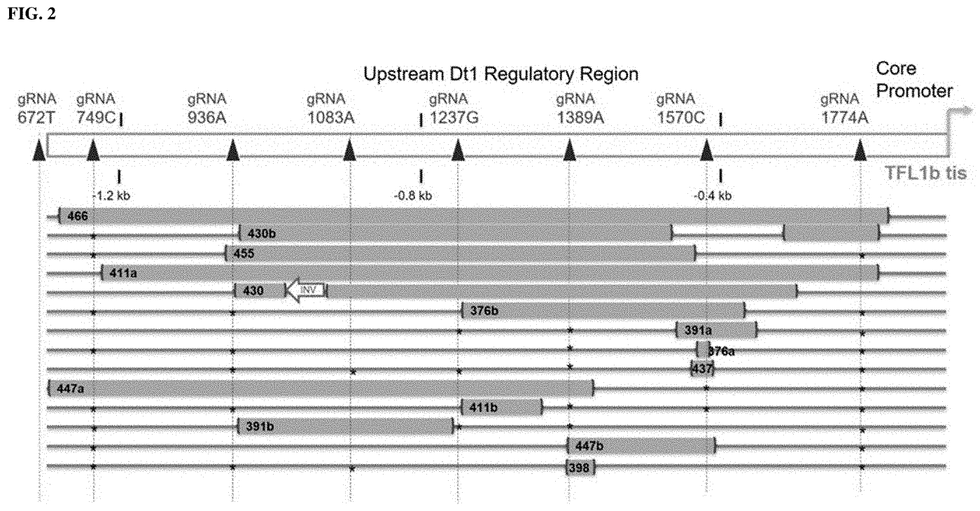

The homozygous edits in Table 2 above are further illustrated in FIG. 2, in alignment with the gRNA spacers illustrated in FIG. 1.

Similarly, soybean plants of maturity group 3.5 were used to produce edited plants using the transformation vectors of the pM205, pM206, and pM207 constructs. Individual R₁ plants produced by selfing R₀ plants having one or more of the edits were assayed for the nature of the edits and the zygosity of the edited mutant or allele and are described in Table 3.

TABLE 3
Edited soybean plants of maturity group 3.5 produced
by the pM205, pM206, or pM207 constructs,
with segmental deletion of the GmTFL1b promoter.
Editing Construct pM205

| | | Causal Deletion | | |
|---|---|---|---|---|
| | | (start and end | | |
| R₀ | | nucleotide positions | R₁ | SEQ |
| Event | Allele Name | of SEQ ID NO: 4) | Zygosity | ID NO |
| S722316 | AL316a | pos. 1552 to pos. 1556 | homozygous | 82 |
| S722316 | AL316b | pos. 1537 to pos. 1565 | homozygous | 83 |
| S722336 | AL336a | pos. 1209 to pos. 1246 | homozygous | 84 |
| S722336 | AL336b | pos. 1209 to pos. 1246; | heterozygous | 85 |
| | | pos. 1552 to pos. 1600 | | |
| S722336 | AL336c | pos. 1553 to pos. 1601 | heterozygous | 86 |

Editing Construct pM207

| | | Causal Deletion | | |
|---|---|---|---|---|
| | | (start and end | | |
| R₀ | | nucleotide positions | R₁ | SEQ |
| Event | Allele Name | of SEQ ID NO: 4) | Zygosity | ID NO |
| S722544 | AL544 | pos. 1368 to pos. 1378 | homozygous | 87 |
| S722589 | AL589a | pos. 1368 to pos. 1395 | homozygous | 88 |
| S722589 | AL589b | pos. 1368 to pos. 1378 | homozygous | 89 |
| S722590 | AL590 | pos. 1344 to pos. 1384 | homozygous | 90 |
| S722601 | AL601 | pos. 1219 to pos. 1453 | homozygous | 91 |
| S722676 | AL676 | pos. 1215 to pos. 1227; | homozygous | 92 |
| | | pos. 1370 to pos. 1376 | | |
| S722702 | AL702a | pos. 1216 to pos. 1376 | homozygous | 94 |
| S722702 | AL702b | pos. 1368 to pos. 1384 | homozygous | 95 |
| S722727 | AL727a | pos. 1366 to pos. 1648 | homozygous | 96 |
| S722727 | AL727b | pos. 1370 to pos. 1489 | heterozygous | 97 |

Editing Construct pM206

| | | Causal Deletion | | |
|---|---|---|---|---|
| | | (start and end | | |
| R₀ | | nucleotide positions | R₁ | SEQ |
| Event | Allele Name | of SEQ ID NO: 4) | Zygosity | ID NO |
| S716698 | AL698 | pos. 1220 to pos. 1556 | homozygous | 93 |
| S716734 | AL734 | pos. 1370 to pos. 1376; | homozygous | 98 |
| | | pos. 1541 to pos. 1615 | | |
| S716766 | AL766a | pos. 1367 to pos. 1375; | homozygous | 99 |
| | | pos. 1551 to pos. 1601 | | |
| S716766 | AL766b | pos. 1371 to pos. 1377 | homozygous | 100 |
| S716787 | AL787 | pos. 1368 to pos. 1553 | homozygous | 101 |
| S716797 | AL797a | pos. 1365 to pos. 1378 | homozygous | 102 |
| S716797 | AL797b | pos. 1365 to pos. 1380; | homozygous | 103 |
| | | pos. 1552 to pos. 1594 | | |
| S716800 | AL800a | pos. 1367 to pos. 1376; | homozygous | 104 |
| | | pos. 1552 to pos. 1682 | | |
| S716800 | AL800b | pos. 1367 to pos. 1376 | homozygous | 105 |
| S716800 | AL800c | pos. 954 to pos. 1391 | homozygous | 106 |

Similarly, soybean plants of maturity group 5.9 were used to produce edited plants using the transformation vectors of the pM204 and pM205 constructs. Individual R₁ plants produced by selfing R₀ plants having one or more of the edits were assayed for the nature of the edits and the zygosity of the edited mutant or allele and are described in Table 4.

TABLE 4
Edited plants of maturity group 5.9
produced by the pM204 or pM205 constructs,
with segmental deletion of the GmTFL1b promoter.
Editing Construct pM205

| | | Causal Deletion | | |
|---|---|---|---|---|
| | | (start and end | | |
| R₀ | | nucleotide positions | R₁ | SEQ |
| Event | Allele Name | of SEQ ID NO: 4) | Zygosity | ID NO |
| S832020 | AL020 | pos. 1535 to pos. 1560 | homozygous | 107 |
| S832025 | AL025a | pos. 1534 to pos. 1644 | homozygous | 108 |
| S832025 | AL025b | pos. 937 to pos. 1599 | homozygous | 109 |
| S832025 | AL025c | pos. 1551 to pos. 1648 | homozygous | 110 |
| S832049 | AL049a | pos. 1526 to pos. 1559 | homozygous | 111 |
| S832049 | AL049b | pos. 1528 to pos. 1606 | homozygous | 112 |
| S832126 | AL126a | pos. 1542 to pos. 1602 | homozygous | 113 |
| S832126 | AL126b | pos. 1214 to pos. 1594 | heterozygous | 114 |
| S737665 | AL665a | pos. 1541 to pos. 1586 | homozygous | 121 |
| S737665 | AL665b | pos. 1552 to pos. 1556 | homozygous | 122 |
| S737665 | AL665c | pos. 669 to pos. 1623 | homozygous | 123 |
| S737746 | AL746a | pos. 1521 to pos. 1558 | homozygous | 124 |
| S737746 | AL746b | pos. 1369 to pos. 1477; | homozygous | 125 |
| | | pos. 1540 to pos. 1554 | | |
| S831991 | AL991a | pos. 1217 to pos. 1674 | homozygous | 126 |
| S831991 | AL991b | pos. 1546 to pos. 1626 | homozygous | 127 |
| S831991 | AL991c | pos. 1547 to pos. 1635 | homozygous | 128 |

Editing Construct pM204

| | | Causal Deletion | | |
|---|---|---|---|---|
| | | (start and end | | |
| R₀ | | nucleotide positions | R₁ | SEQ |
| Event | Allele Name | of SEQ ID NO: 4) | Zygosity | ID NO |
| S737331 | AL331a | pos. 1368 to pos. 1554 | homozygous | 115 |
| S737331 | AL331b | pos. 1369 to pos. 1477; | homozygous | 116 |
| | | pos. 1550 to pos. 1554 | | |
| S737331 | AL331c | pos. 734 to pos. 2000 | homozygous | 117 |
| S737398 | AL398a | pos. 1371 to pos. 1560 | homozygous | 118 |
| S737398 | AL398b | pos. 1370 to pos. 1376 | homozygous | 119 |
| S737398 | AL398c | pos. 1370 to pos. 1376; | homozygous | 120 |
| | | pos. 1537 to pos. 1572 | | |

### Example 3. GmTFL1b Gene Expression Levels in Modified Soybean Plants

R₂ plants having homozygous alleles of GmTFL1b promoter mutants (Dt1 alleles) and wild-type control plants were selected and grown in a controlled environment for sampling to assess differences in expression of the target gene. Plants were sampled at VC stage, which is defined as the period after the emergence stage but before the V1 stage, when cotyledons and unifoliates are fully expanded. Unifoliate leaf and apex tissues were collected on dry ice for gene expression analysis by TaqMan assay. Ten biological replicates were measured per allele entry.

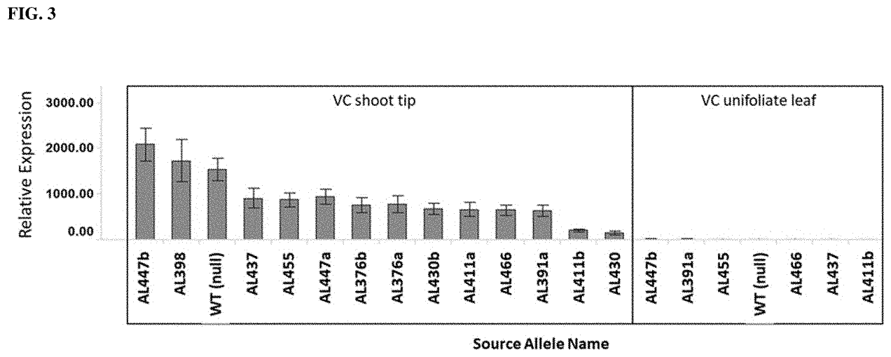

Table 5 and FIG. 3 show the relative expression levels of the GmTFL1b gene in leaf and apex tissues at VC stage, of edited soybean plants with various homozygous Dt1 alleles. The edited plants were produced using editing construct pM552. As shown in FIG. 3, expression of GmTFL1b in leaf tissue was nominal for all samples tested.

TABLE 5
Relative expression levels of GmTFL1b gene
in apex tissues at VC stage in edited soybean
plants with various homozygous Dt1 alleles.

| | | Causal Lesion | | |
|---|---|---|---|---|
| | | (start and end | Relative | |
| R₀ | | nucleotide positions | Expression Level | |
| Event | Allele Name | of SEQ ID NO: 4) | Mean | STD Error |
| S376 | Null (WT) | N/A | 1532.01 | 180.12 |
| S376 | AL376a | pos. 1539 to pos. 1568 | 785.74 | 117.73 |
| S376 | AL376b | pos. 1217 to pos. 1604 | 761.69 | 129.95 |
| S391 | AL391a | pos. 1518 to pos. 1629 | 533.60 | 78.67 |
| S391 | AL391b | pos. 951 to pos. 1222 | nd | nd |
| S398 | AL398 | pos. 1364 to pos. 1407 | 1728.11 | 313.12 |
| S410 | AL410a | pos. 1367 to pos. 1516 | nd | nd |
| S411 | AL411a | pos. 754 to pos. 1806 | 665.83 | 114.64 |
| S411 | AL411b | pos. 1216 to pos. 1319 | 195.73 | 22.74 |
| S411 | AL411c | pos. 1159 to pos. 1965 | nd | nd |
| S419 | AL419 | pos. 760 to pos. 1214 | nd | nd |
| S430 | AL430a | pos. 939 to pos. 1028; | 138.85 | 29.66 |
| | | INV-pos. 1065 to pos. 1029 | | |
| | | pos. 1066 to pos. 1664 | | |
| S430 | AL430b | pos. 952 to pos. 1552; | 673.69 | 96.28 |
| | | pos. 1677 to pos. 1808 | | |
| S437 | AL437 | pos. 1524 to pos. 1558 | 909.13 | 151.91 |
| S447 | AL447a | pos. 476 to pos. 1405 | 946.29 | 111.52 |
| S447 | AL447b | pos. 1365 to pos. 1559 | 2087.50 | 288.51 |
| S455 | AL455 | pos. 928 to pos. 1560 | 873.15 | 108.95 |
| S461 | AL461 | pos. 346 to pos. 2092 | nd | nd |
| S466 | AL466 | pos. 593 to pos. 1813 | 650.28 | 80.61 |

As the promoter was mutated by genome editing, most of the Dt1 alleles led to reduced expression level of GmTFL1b, in comparison with wild-type plants having the endogenous promoter. However, mutant alleles AL398 and AL447b, exhibited increased gene expression levels. Expression of TFL1 was greatly reduced for all but two of the samples tested.

### Example 4. Evaluation of Terminal Flowering Date and Maturity Date Phenotypes of Plants Comprising Edited Alleles

To study phenotypes of the edited plants, R2 plants having homozygous alleles of GmTFL1b promoter mutants (Dt1 alleles) and wild-type control plants were selected and grown in the covered nursery and in the field under standard agronomic practice.

All edited plants showed emergence as good as the wild-type or better, even the plants of near-determinate AL430a allele. All edited plants initiated flowering like the wild-type indeterminate control. No detectable change in floral initiation was observed for edited plants, indicating that stem growth habit traits controlled by the Dt1 locus do not interact with genes that control floral initiation.

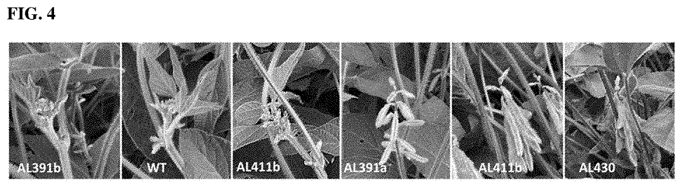

As observed in covered nurseries, plants with edited Dt1 alleles displayed a range of determinacy phenotypes ranging from near-determinate to more indeterminate than wild-type (extreme indeterminacy), with several distinct varieties of semi-determinate behavior, first evidenced by differential terminal flowering date as shown in FIG. 4. The terminal flowering date is defined as the date on which there is one open flower at the terminal node of the main stem on 50% of the plants in a uniform plot segment. Plots in this evaluation consisted of 2 rows 5′ in length with 30″ spacing between them; about 80 seeds sown per plot; plots on a row had 1.5′ alleys between them.

Lodging in soybean plots is evaluated through multiple observations beginning at the R6 developmental stage using a rating scale to determine the portion of each plot that exhibits stem lodging. Observations continue through senescence at late R7 developmental stage. Plots are rated for lodging using the scale in Table 6.

TABLE 6
Lodging score determination in soybean plots.

| Score | Phenotype |
|---|---|
| 1 | 90 to 100% of the plants are standing straight |
| 2 | 80 to 89% of the plants are standing straight |
| 3 | 70 to 79% of the plants are standing straight |
| 4 | 60 to 69% of the plants are standing straight |
| 5 | 50 to 59% of the plants are standing straight |
| 6 | 40 to 49% of the plants are standing straight |
| 7 | 30 to 39% of the plants are standing straight |
| 8 | 20 to 29% of the plants are standing straight |
| 9 | 0 to 19% of the plants are standing straight |

Terminal flowering date, maturity date, and lodging rating were determined for wild-type plants and plants of homozygous edited alleles in observation plots. Terminal flowering date is calculated here as the number of calendar days since January 1st of the year of testing. The maturity date is defined as the date when 95% of the pods in a plot have reached their mature pod color. Where the precise maturity date was not observed, a “>” or “<” sign is provided to indicate that the onset of maturity date is earlier than or later than the listed date. Terminal flowering dates and maturity dates are determined through sequential observation of experimental plots until all plots have achieved the developmental stage. Scoring of developmental stages was conducted at plot level on a predetermined 20″ internal segment of the plot that is uniform and undamaged (containing about 15 consecutive plants). A plot is considered to have reached the developmental stage in question when >50% of plants in the measured plot segment are at that stage. Changes in determinacy can be quantified in terms of days from planting to terminal flowering (DOTF). In a field with a given planting date which is the same for all plants in testing, the terminal flowering date and other phenology characteristics can be recorded by the calendar day of the year for simple calculation of differences from the control. Observations from the plants in the field environment indicate that edited alleles of GmTFL1b gene promoter region resulted in diverse range of terminal flowering dates extending from 22 days prior to that of wild-type indeterminate plants to 4 days later, as shown in Table 7. The edited plants were produced using the pM552 editing construct.

TABLE 7
Values for terminal flowering date (TF),
maturity date (R8), and lodging rating.

| | | | | | Relative |
|---|---|---|---|---|---|
| | | | | Lodging | Expression |
| R₀ | | TF (# days | | Rating | Level |
| Event | Allele | since Jan. 1) | R8 | (1-9) | (mean) |
| S376 | Null (WT) | 214 | 265 | 6 | 1532.01 |
| S398 | AL398 | 218 | >267 | 9 | 1728.11 |
| S391 | AL391b | 216 | 267 | 6 | ND |
| S447 | AL447b | 216 | 267 | 7 | 2087.5 |
| S447 | AL447a | 212 | 265 | 7 | 946.29 |
| S455 | AL455 | 211 | 265 | 3 | 873.15 |
| S411 | AL411a | 207 | 265 | 3 | 665.83 |
| S391 | AL391a | 203 | 265 | 1 | 533.6 |
| S411 | AL411b | 199 | <265 | 1 | 195.73 |
| S430 | AL430a | 192 | <265 | 1 | 138.85 |

As shown in Table 7, changes in terminal flowing dates were not accompanied by any delay in R8 maturity date for most edited plants, except for plants having alleles AL398, AL391b, and AL447b, all of which have a later terminal flowing date than unedited wild-type plants, and thus more indeterminate than the wild-type plants.

It is expected that plants of stronger determinacy, or earlier terminal flowering date would produce architectural features making the plants more resistant to lodging. Indeed, as shown in Table 7, there is a clear trend toward reduced lodging in plants with an earlier terminal flowering date. Through genome editing in the promoter region of the GmTFL1b gene, plants with a range of terminal flowering dates were produced, corresponding to varying levels of semi-determinacy, leading to reduction or elimination of the risk of lodging. Lodging accounts for lost harvest value, particularly with taller elite soybean varieties. Reduction of lodging has the potential for increased soy harvest value and yield. Reduced lodging or increased standability may also improve the ease of access to the field at all points following canopy closure, providing benefits such as effective fertilization and pest control.

The mean relative expression level of GmTFL1b gene is copied from Table 5 to Table 7. As the endogenous GmTFL1b gene in indeterminate soybeans is the dominant allele for the indeterminacy trait, reduction in the expression level of the GmTFL1b gene would lead to reduction in the level of indeterminacy (or an increase in determinacy). As shown in Table 7, lower mean relative expression levels tend to correspond to earlier terminal flowering date, or stronger determinacy. This correspondence indicates that expression level of GmTFL1b gene can be used as a predictor for terminal flowering date or level of determinacy of edited plants.

Differences in relative expression level correlate not only with loss-of-function mutants but also with gain-of-function in indeterminacy. Gain-of-function mutants exhibit extreme indeterminacy as compared to wild-type plants, as well as increased expression consistent with creation of hypermorphic alleles through genome editing of the promoter region of the GmTFL1b gene.

### Example 5. Yield Potential Estimate by Derived from Pod Number Per Plant

As shown in Example 4, edited soybean plants with an early terminal flowing date phenotype tended to have improved lodging and standability characteristics. However, an early terminal flowing date could also result in lost yield due to premature termination of main stem and branches, reducing the number of productive nodes on the plant to such an extent that any improvement in nodal efficacy achieved by earlier assertion of apical dominance and full dedication of source to reproductive development is unable to offset. It is worth noting that significantly earlier terminal flowing date than wild-type with retention of determinate apical node productivity can be sustained without substantially changing plant height (and likely total node number). This means that the per plant pod yield may not be reduced by introduction of the semi-determinate trait, while still reaping the benefits of increased standability.

Pod number per plant is determined at plot level on a predetermined 20″ internal segment of the plot that is uniform and undamaged (containing about 15 consecutive plants). At R6 stages or later, all plants from the predetermined segment of each plot are cut and packaged to ensure no pods are lost. Pods from each bundle are systematically counted and recorded. Pod number per entry (allele) is reported as the mean of all counted plants per entry. Plants comprising edited alleles AL391a and AL411a, which demonstrated optimal lodging characteristics (see Table 7), and wild-type indeterminate plants were evaluated for pod number per plant and the preliminary results are shown in Table 8 below.

TABLE 8
Pod number per plant values for wild-type and
edited plants with optimal lodging characteristics.

| R₀ Event ID | Allele | Mean Pods/Plant | STD Error |
|---|---|---|---|
| S376 | Null (WT) | 45.6 | 18.7 |
| S391 | AL391a | 64.5 | 16.1 |
| S411 | AL411a | 59.4 | 20 |

The edited plants were produced using the pM552 editing construct. The pod numbers per plant are comparable among the 3 plant groups, within the margin of error. The edited semi-determinate plants demonstrated increased standability with no loss of pods. In fact, the mean pod counts for edited semi-determinate plants are greater than for the wild-type indeterminate plants, suggesting that introducing semi-determinacy can result in increased productivity along with reducing yield loss through reduced lodging.

Since the Dt1 locus was fixed early during soybean domestication, these edited alleles may be recapitulated in any indeterminate soybean germplasm, conferring semi-determinacy and resulting traits on any cultivated line. Significant value may be created through increased standability and productivity across soybean acres, particularly in high yielding environments. In addition, the more compact plant type resulting from semi-determinate traits may facilitate growth in narrow rows and even higher densities. Under these intensive agronomic configurations, the compact, upright plant type should permit improved airflow under the canopy, thereby reducing humidity and with this, the potential for decreased fungal disease susceptibility, including reduced susceptibility to economically important white mold and sudden death syndrome (SDS).

### Example 6. Gene Editing of TFL1 Homologs

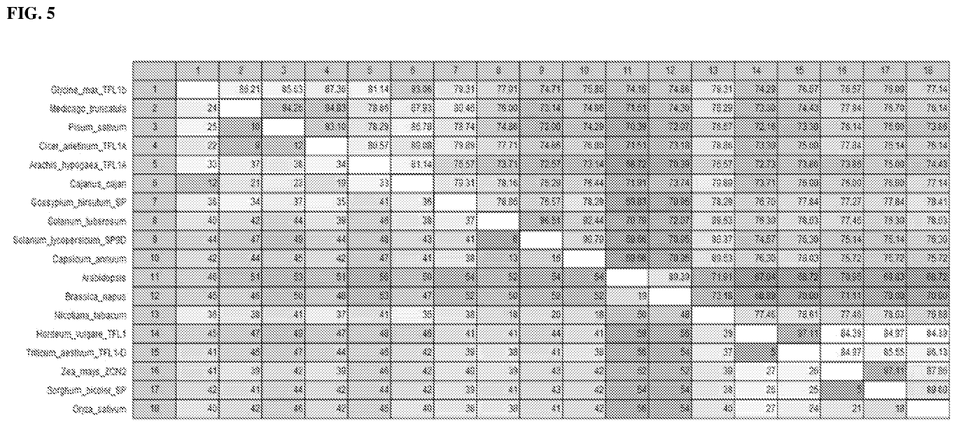

A subset of agriculturally relevant plant species were selected for sequence comparison based on total harvest value and diversity, including members of the Solanaceae, Brassicaceae, and Leguminae families, plus cotton and monocot cereals. To identify the polypeptide sequence hit from each species with the highest identity, NCBI's BLASTP program was used with the soybean TFL1b (Dt1) polypeptide sequence as the query sequence and the non-redundant sequence collection database as the search set. The identified sequences were aligned by CLUSTAL type algorithm in CLC Workbench (default parameters for very accurate alignment). The pairwise comparisons are provided in tabular form (FIG. 5).

The polynucleotide sequences of the 2 kb promoter region upstream of the transcription initiation site (tis) of the TFL1b gene homologs found in Zea mays, Sorghum bicolor, Oryza sativa, Triticum aestivum, Hordeum vulgare, Solanum lycopersicum, Gossypium hirsutum, Capsicum annuum, Brassica napus, Arabidopsis thaliana, Medicago truncatula, Arachis hypogaea, and Cicer arietinum are provided herein as SEQ ID NOs:67-77, 79, and 81, respectively. In order to create plants having modified determinacy phenotypes in these additional plant species or in other plant species, a procedure similar to that described above in Example 1 can be used.

Following a similar process as described above in Example 1, approximately ten editing guide RNAs can be designed in approximately equal spacing over the approximately 2 kb promoter region the TFL1b homolog (see FIG. 1 for illustration), following common gRNA design constraints. The basal promoter region of approximately 200 nucleotides located at the 3′ end of the sequences of SEQ ID NOs:67-77, 79, and 81 can be avoided to minimize the chance of rendering the promoter completely nonfunctional. Following the gRNA design, steps in line with those described in Example 1 can be followed to create editing constructs to produce edited plants, which can be further subjected to molecular and phenotypic characterization to select for desired level of relative expression and modulation of determinacy.

### Example 7. Evaluation of Additional Phenotypic Characteristics of Plants Comprising Edited Dt1 Alleles

Additional phenotypic characteristics of the soybean plants comprising edited Dt1 alleles were evaluated. All trait effects described in this example are reported as delta of the mean value for a given entry versus the indeterminate wild-type plants (unmodified soybean plants of maturity group 3.5).

Plots contained 4 rows that were 10 feet in length with 20-inch spacing between them. Each range of plots had a 3′ alley between them. A total of 288 seeds were sown per plot. Each test entry was replicated in 12 plots per experiment, with randomized mapping in each test entry replication set which included sufficient plots containing wild-type comparator plants. Trait metrics described below were collected for each plot on the field. The resulting trait values reported are the delta of means, the difference between the test mean and the comparison mean, for all 12 replicates. For this experimental design, a P-value <0.2 is considered statistically significant. Scoring of developmental stages was conducted at plot level on a predetermined 20-inch internal segment of the plot that was uniform and undamaged (containing about 15 consecutive plants). A plot was considered to have reached a specific developmental stage when ≥50% of plants in the measured plot segment were at that stage. For large scale quantitative comparison, stage determination is reported in terms of the number of days from planting to the desired stage.

Plant development and morphology traits, including full maturity date, number of branches per plant, plant height, and plant lodging (standability) were evaluated at the R8 developmental stage. Full maturity was determined based on the number of days from the planting date to the date on which 95% of the pods on a plant have reached their mature pod color. Plant height was recorded as the direct measurement of linear main stem length. Plant lodging was evaluated as described in Example 4 above. The results are shown in Table 9 below.

TABLE 9
Year 2 field data for plant development and morphology at R8 growth stage for
plants comprising homozygous edited Dt1 alleles.

| | Full Maturity | | # Branches per Plant | | Plant Height | | Plant Lodging | |
|---|---|---|---|---|---|---|---|---|
| | (WT mean = 123 Days) | | (WT mean = 2.1) | | (WT mean = 37.9 in) | | (WT mean = 5.8) | |
| | Mean Δ | | Mean Δ | | Mean Δ | | Mean Δ | |
| Allele | (Days) | P-value | (Count) | P-value | (Inches) | P-value | (Rating) | P-value |
| AL391a | −0.9 | 0.08 | 0.2 | 0.55 | −7.79 | 0.00 | −4.7 | 0.00 |
| AL430a | 0.0 | 0.94 | 4.3 | 0.00 | −29.47 | 0.00 | −4.4 | 0.00 |
| AL411b | −0.4 | 0.41 | 0.4 | 0.08 | −20.51 | 0.00 | −4.3 | 0.00 |
| AL376a | −0.5 | 0.39 | 0.0 | 0.87 | −6.65 | 0.00 | −4.2 | 0.00 |
| AL437 | −1.1 | 0.07 | −0.8 | 0.00 | −6.52 | 0.00 | −3.8 | 0.00 |
| AL376b | −0.7 | 0.33 | 0.1 | 0.74 | −8.22 | 0.00 | −3.8 | 0.00 |
| AL411a | −0.6 | 0.23 | 0.0 | 0.88 | −5.06 | 0.00 | −3.7 | 0.00 |
| AL430b | −0.5 | 0.38 | −0.2 | 0.31 | −3.71 | 0.03 | −2.9 | 0.00 |
| AL466 | −0.8 | 0.18 | −0.4 | 0.16 | −4.25 | 0.02 | −2.7 | 0.00 |
| AL455 | −0.3 | 0.49 | −0.2 | 0.37 | −2.10 | 0.12 | −2.0 | 0.00 |
| AL447b | −0.7 | 0.13 | 0.1 | 0.65 | −2.97 | 0.04 | −1.4 | 0.01 |
| AL391b | −0.4 | 0.42 | −0.1 | 0.60 | 0.09 | 0.94 | −1.0 | 0.07 |
| AL447a | 1.0 | 0.05 | 0.3 | 0.12 | 1.05 | 0.40 | 1.6 | 0.00 |
| AL398 | 3.0 | 0.00 | 0.1 | 0.67 | 3.18 | 0.03 | 1.9 | 0.00 |

* Data for all alleles is ordered by plant lodging values, from lowest to highest.

As shown in Table 7 of Example 4 above, plants carrying extreme indeterminate alleles (exhibiting increased GmTFL1b gene expression levels), such as AL398, had delayed termination along with significantly delayed maturity. However, as shown in Table 9 above, delayed termination in plants comprising AL398 alleles was accompanied by a modestly higher branch number, increased plant height, and increased lodging. All plants carrying semi-determinate alleles matured within 1 day of the indeterminate wild-type plants, with the exception of plants carrying AL437 alleles, which matured over a day earlier than the indeterminate wild-type plants. Plants containing the semi-determinate alleles had significant reductions in plant height, ranging from 2 inches to 30 inches, compared to the indeterminate wild-type plants. Plants containing the semi-determinate alleles were more standable, exhibiting lodging ratings ranging from 1-4.7 points (10%-47%) lower than the wild-type indeterminate plants. This is due to, in part, the reduction in plant height. Despite decreases in plant height and lodging, most semi-determinate alleles enabled plants to bear a number of branches per plant that was not statistically different than the indeterminate wild-type plants. Only plants carrying allele AL437 had a substantially reduced branch number.

Secondary yield components including node number per plant, pod number per plant, and pods per node were also evaluated. Secondary yield components were determined at plot level on a predetermined 20-inch internal segment of the plot that was uniform and undamaged (containing about 15 consecutive plants). At the R8 stage, all plants from the predetermined segment of each plot were cut and packaged to ensure no pods were lost. Pod number and node number on each plant was systematically counted and recorded. Plant number was recorded and pods from a given plot were combined for a primary yield component analysis. Pod number per node was calculated from counted values. The results are shown in Table 10 below.

TABLE 10
Year 2 field data for secondary yield components at R8 growth stage for plants
comprising homozygous edited Dt1 alleles.

| | Nodes per Plant | | Pods per Node | | Pods per Plant | |
|---|---|---|---|---|---|---|
| | (WT mean = 34.8) | | (WT mean = 2.0) | | (WT mean = 68.0) | |
| | Mean Δ | | Mean Δ | | Mean Δ | |
| Allele | (Count) | P-value | (Count) | P-value | (Count) | P-value |
| AL391a | 1.9 | 0.42 | 0.30 | 0.05 | 14.1 | 0.01 |
| AL430a | −1.5 | 0.48 | −0.65 | 0.00 | −22.8 | 0.00 |
| AL411b | −2.2 | 0.22 | −0.14 | 0.18 | −8.9 | 0.03 |
| AL376a | −0.9 | 0.71 | 0.30 | 0.06 | 8.5 | 0.14 |
| AL437 | −0.7 | 0.75 | 0.04 | 0.75 | 0.4 | 0.94 |
| AL376b | −2.4 | 0.52 | 0.86 | 0.00 | 23.0 | 0.01 |
| AL411a | −0.3 | 0.86 | 0.32 | 0.01 | 10.4 | 0.01 |
| AL430b | −2.8 | 0.18 | 0.12 | 0.30 | −1.0 | 0.84 |
| AL466 | −3.3 | 0.13 | 0.13 | 0.29 | −0.6 | 0.90 |
| AL455 | −1.0 | 0.61 | 0.32 | 0.02 | 9.3 | 0.03 |
| AL447b | 0.7 | 0.68 | −0.05 | 0.57 | 0.2 | 0.96 |
| AL391b | 0.8 | 0.67 | −0.11 | 0.29 | −1.1 | 0.80 |
| AL447a | 3.8 | 0.03 | −0.29 | 0.01 | −3.1 | 0.42 |
| AL398 | 2.0 | 0.25 | 0.03 | 0.73 | 4.8 | 0.23 |

* Data for all alleles is ordered by plant lodging values, from lowest to highest.

Plants carrying semi-determinate alleles having significantly earlier termination in the field also exhibited significantly improved standability in the field with no loss of branch number and no change in maturity greater than one day. These characteristics suggest that semi-determinate alleles may improve standability without decreased productivity. However, earlier termination of stem tips could also result in lost yield by reducing the number of productive nodes on the plant to such an extent that any improvement in nodal efficacy achieved by earlier assertion of apical dominance and full dedication of source to reproductive development is unable to offset. Although plants carrying the semi-determinate alleles do exhibit some degree of reduction in plant height, depending upon the allele, these reductions are not generally associated with significant changes in productive nodes per plant. Consistent with node retention, most semi-determinate alleles confer pod numbers per plant that are at parity with or significantly higher than the pod numbers per plant counted for indeterminate wild-type plants. Pods per plant for most semi-determinate plants tested range from equivalency up to 23 pods higher than the indeterminate wild-type plants. Increased pod numbers for semi-determinate plants were associated with improved nodal efficacy, indicated by significantly higher pod numbers per node. The exceptions were the AL430a and AL411b alleles, which had significantly negative pod trait metrics that resulted from an extreme reduction in plant height due to the extent of early termination and near loss of Dt1 function as shown in Table 8 earlier.

Primary yield components including seeds per plant, thousand seed weight, and grain yield estimate were also evaluated. Primary yield components were determined at plot level by threshing pods collected from all plants in a given plot and normalizing by the number of plants measured. Threshing returns total seed weight and seed number. Thousand seed weight per plot is calculated by dividing total seed weight by seed number and multiplying by 1000. Grain yield estimate is a function of estimated seeds per unit area multiplied by single seed weight multiplied by the conversion factor 45.375, returning an estimate for relative comparison only, in terms of 60 pound bushels per acre. The results are shown in Table 11 below.

TABLE 11
Year 2 field data for primary yield components at R8 growth stage for plants
comprising homozygous edited Dt1 alleles.

| | Seeds per Plant | | Thousand Seed Weight | | Grain Yield Estimate | |
|---|---|---|---|---|---|---|
| | (WT mean = 158.9) | | (WT mean = 6.02 oz) | | (WT mean = 99.7 bu/ac) | |
| | Mean Δ | | Mean Δ | | Mean Δ | |
| Allele | (Count) | P-value | (oz/1000) | P-value | (bu/ac) | P-value |
| AL391a | 12.6 | 0.33 | −0.53 | 0.00 | 0.28 | 0.97 |
| AL430a | −64.6 | 0.00 | 0.04 | 0.78 | −41.97 | 0.00 |
| AL411b | −38.3 | 0.00 | −0.10 | 0.34 | −19.40 | 0.00 |
| AL376a | 10.7 | 0.46 | −0.59 | 0.00 | −0.01 | 1.00 |
| AL437 | −12.9 | 0.27 | −0.99 | 0.00 | −18.77 | 0.01 |
| AL376b | 26.5 | 0.19 | −0.62 | 0.01 | −1.38 | 0.91 |
| AL411a | 15.4 | 0.12 | −0.34 | 0.00 | 7.59 | 0.23 |
| AL430b | −15.4 | 0.17 | −0.33 | 0.01 | −4.96 | 0.49 |
| AL466 | −8.6 | 0.46 | −0.43 | 0.00 | −11.33 | 0.13 |
| AL455 | 14.3 | 0.18 | −0.34 | 0.00 | 3.97 | 0.55 |
| AL447b | −1.6 | 0.87 | −0.25 | 0.01 | −2.33 | 0.69 |
| AL391b | −0.2 | 0.98 | 0.15 | 0.17 | 3.26 | 0.60 |
| AL447a | −6.3 | 0.50 | 0.13 | 0.21 | 2.34 | 0.69 |
| AL398 | 2.2 | 0.82 | −0.80 | 0.00 | −9.05 | 0.13 |

* Data for all alleles is ordered by plant lodging values, from lowest to highest.

The secondary yield components observed for most semi-determinate alleles described herein were either at parity or significantly positive compared to the indeterminate wild-type plants. These characteristics indicate that significant improvement in standability of soybeans can be achieved by editing without yield loss. Alleles with positive secondary yield components also exhibited increased seeds per plant in the field in many cases. Most semi-determinate alleles showed some reduction in seed weight in these experiments. However, despite the reduction in seed weight, all plants carrying semi-determinate alleles with positive seed numbers still showed neutral to positive grain yield estimates. Alleles AL411b and AL430a, which were previously noted to have significantly negative pod trait metrics resulting from an extreme reduction in plant height, also showed reductions in seeds per plant and grain yield estimate. This indicates the useful limit in trait magnitude to reduction of stem tip termination. These observations provide substantial support for the potential of use of alleles conferring semi-determinacy of the appropriate magnitude to increase standability as well as yield potential in some cases.

The following above-ground traits were evaluated: canopy coverage rating, number of days from planting to R1 stage (beginning flower), and number of days from planting to terminal pod (TP). Canopy coverage rating represents the percentage of a plot area that is occupied by vegetation. The evaluation is made based on remote RGB imaging data acquired by unmanned aerial vehicles that distinguishes green pixels from non-green pixels in a plot, as viewed from directly above the plot. For example, a canopy coverage rating of 40% indicates that 60% of the plot's planted space is likely unoccupied. Termination was measured by observation of the date of terminal flower or terminal pod formation. R1 stage and terminal pod stage durations were determined through sequential observation of experimental plots until all plots had achieved the stated developmental stage. Floral initiation was measured by noting the date on which there was an open flower at any node on the main stem of the plant. Terminal pod was measured by noting the date on which there was one pod ≥ 3/16 inch (0.5 cm) long on the terminal node of the main stem. The results are shown in Table 12 below.

TABLE 12
Year 3 field data for above-ground traits for plants comprising
homozygous edited Dt1 alleles.

| | Canopy Cover Rating | | # Days-Planting to R1 | | # Days-Planting to TP | |
|---|---|---|---|---|---|---|
| | (WT mean = 33.93) | | (WT mean = 35.6 Days) | | (WT mean = 69.1 Days) | |
| | Mean Δ | | Mean Δ | | Mean Δ | |
| Allele | (%) | P-value | (days) | P-value | (days) | P-value |
| AL391a | 0.17 | 0.79 | −0.8 | 0.01 | −12.00 | 0.00 |
| AL411a | 0.62 | 0.32 | −1.0 | 0.00 | −9.75 | 0.00 |
| AL411b | −1.11 | 0.08 | −1.0 | 0.00 | −12.08 | 0.00 |
| AL376b | −0.55 | 0.38 | −0.3 | 0.31 | −12.08 | 0.00 |
| AL410a | 0.23 | 0.70 | −1.1 | 0.00 | −9.17 | 0.00 |
| AL430b | 0.93 | 0.14 | −1.0 | 0.00 | −8.67 | 0.00 |
| AL437 | −2.39 | 0.00 | −2.3 | 0.00 | −9.25 | 0.00 |
| AL455 | 0.91 | 0.15 | −0.6 | 0.04 | −3.50 | 0.00 |

* Data for all alleles is ordered by plant lodging values, from lowest to highest.

The primary trait impacted by edits to the Dt1 locus is termination date. The spectrum of changes in termination traits resulting from editing the Dt1 locus bring about proportional downstream changes in plant height and standability. Significant reductions in the number of days from planting to terminal pod for semi-determinate alleles, ranging from 3.5 to 12 days earlier than the indeterminate wild-type plants, are associated with minimal changes in floral initiation and maturity. Plants carrying allele AL437 initiated flowering within 1 day of the indeterminate wild-type plants. Dt1 editing created statistically significant changes in termination of different magnitudes depending upon the semi-determinate allele. The above-ground traits for most semi-determinate edited plants measured did not differ significantly from the indeterminate wild-type plants at the R1 growth stage.

The below-ground traits of root dry weight, shoot dry weight, and shoot to root ratio related to biomass were also measured at the R1 stage. These traits were evaluated at plot level on a predetermined 20-inch internal segment of the plot that was uniform and undamaged (containing about 15 consecutive plants). At the R1 stage, all plants from the predetermined segment of each plot were removed from the ground whole, counted, and divided into shoot and root fractions and packaged for transfer to the oven. Plant samples were dried to completion and then weights were recorded by plot. Root and shoot biomass were reported on a mean per plant basis. Shoot to root ratio was calculated by dividing mean shoot dry weight by mean root dry weight for each entry. The results are shown in Table 13 below.

TABLE 13
Year 3 field data for below-ground traits at R1 growth stage for plants comprising
homozygous edited Dt1 alleles.

| | Root Dry Weight | | Shoot Dry Weight | | Shoot:Root Ratio | |
|---|---|---|---|---|---|---|
| | (WT mean = 0.12 oz) | | (WT mean = 0.28 oz) | | (WT mean = 2.34) | |
| | Mean Δ | | Mean Δ | | Mean Δ | |
| Allele | (oz) | P-value | (oz) | P-value | (ratio) | P-value |
| AL391a | 0.005 | 0.34 | 0.004 | 0.84 | −0.06 | 0.64 |
| AL411a | 0.004 | 0.48 | 0.023 | 0.20 | 0.13 | 0.33 |
| AL411b | −0.005 | 0.34 | 0.021 | 0.25 | 0.29 | 0.04 |
| AL376b | −0.005 | 0.29 | −0.004 | 0.83 | 0.15 | 0.31 |
| AL410a | −0.005 | 0.30 | −0.007 | 0.69 | 0.07 | 0.62 |
| AL430b | 0.003 | 0.60 | 0.006 | 0.72 | −0.02 | 0.88 |
| AL437 | −0.007 | 0.16 | −0.055 | 0.00 | −0.33 | 0.02 |
| AL455 | 0.005 | 0.30 | 0.017 | 0.35 | 0.06 | 0.70 |

* Data for all alleles is ordered by plant lodging values, from lowest to highest.

Below-ground biomass measurements and ratios for most plants carrying semi-determinate alleles did not differ significantly from the indeterminate wild-type plants at the R1 growth stage. One of the semi-determinate alleles, AL437, was an exception and was significantly negative for all three biomass metrics, indicating a smaller overall plant size with a disproportionately smaller shoot. This is apparent from a significantly smaller canopy coverage rating for this allele, as shown in Table 12.

Overall, these trait results indicate the utility of editing for semi-determinacy to obtain unique alleles that are the basis for more standable soybean varieties with stable or improved yield potential. In addition, the results show that editing of the Dt1 locus can generate not only semi-determinate alleles, but also near-determinate, determinate, and extreme indeterminate alleles.

Having described the present disclosure in detail, it will be apparent that modifications, variations, and equivalent embodiments are possible without departing from the spirit and scope of the present disclosure as described herein and in the appended claims. Furthermore, it should be appreciated that all examples in the present disclosure are provided as non-limiting examples.

1. A modified plant, plant seed, plant part, or plant cell, comprising a modification that reduces the expression or activity of TFL1, or a homolog thereof, as compared to the expression or activity of TFL1 or the homolog thereof in an otherwise identical plant, plant seed, plant part, or plant cell that lacks the modification.

2. The modified plant, plant seed, plant part, or plant cell of claim 1, wherein:
(a) the modification is present in at least one allele of an endogenous TFL1 gene or homolog thereof;
(b) the plant, plant seed, plant part, or plant cell is heterozygous for the modification;
(c) the plant, plant seed, plant part, or plant cell is homozygous for the modification;
(d) the plant, plant seed, plant part, or plant cell comprises a first modification in a first allele of the TFL1 gene and a second modification in a second allele of the TFL1 gene, the first modification and the second modification being different from one another; or
(e) the plant is a leguminous plant, a cotton plant, or a canola plant; or wherein the plant seed, plant part, or plant cell is a plant seed, plant part, or plant cell of a leguminous plant, a cotton plant, a canola plant, a corn plant, a sorghum plant, a rice plant, a wheat plant, a barley plant, a tomato plant, or a pepper plant.

3. The modified plant, plant seed, plant part, or plant cell of claim 2, wherein the TFL1 gene or homolog thereof encodes a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO:2.

4. The modified plant, plant seed, plant part, or plant cell of claim 2, wherein the modification is in a non-coding region of the TFL1 gene or homolog thereof.

5. The modified plant, plant seed, plant part, or plant cell of claim 4, wherein the non-coding region is selected from the group consisting of a promoter, an intron, a 5′-untranslated region, a 3′-untranslated region, and combinations of any thereof.

6. The modified plant, plant seed, plant part, or plant cell of claim 5, wherein the modification is in the promoter of the TFL1 gene or homolog thereof.

7. (canceled)

8. The modified plant, plant seed, plant part, or plant cell of claim 2, wherein the plant is a leguminous plant, or wherein the plant seed, plant part, or plant cell is a plant seed, plant part, or plant cell of a leguminous plant.

9. The modified plant, plant seed, plant part, or plant cell of claim 8, wherein the leguminous plant is a soybean plant, a bean plant, a pea plant, a chickpea plant, an alfalfa plant, a peanut plant, a carob plant, a lentil plant, or a licorice plant.

10. The modified plant, plant seed, plant part, or plant cell of claim 9, wherein the leguminous plant is a soybean plant.

11. The modified plant, plant seed, plant part, or plant cell of claim 10, wherein the TFL1 gene is a TFL1b gene.

12-14. (canceled)

15. The modified plant, plant seed, plant part, or plant cell of claim 1, wherein:
(a) the modification comprises a deletion, an insertion, a substitution, an inversion, or a combination of any thereof;
(b) the modification alters the expression level of the TFL1 protein, as compared to the expression level of the TFL1 protein in an otherwise identical plant, plant seed, plant part, or plant cell that lacks the modification;
(c) the modification alters the determinacy phenotype of the plant, as compared to the determinacy phenotype of an otherwise identical plant that lacks the modification
(d) after planting, the modified plant reaches its terminal flowering date sooner than an otherwise identical plant that lacks the modification;
(e) the modified plant exhibits a reduced lodging rate, as compared to the lodging rate of an otherwise identical plant that lacks the modification;
(f) the modified plant exhibits substantially the same or increased yield, as compared to the yield of an otherwise identical plant that lacks the modification; or
(g) the modified plant exhibits lower susceptibility to fungal disease, as compared to the susceptibility of an otherwise identical plant that lacks the modification.

16. The modified plant, plant seed, plant part, or plant cell of claim 15, wherein the modification is located at about 200 nucleotides or more from the 3′ end of a sequence selected from the group consisting of SEQ ID NOs:4, 67-77, 79, and 81.

17. The modified plant, plant seed, plant part, or plant cell of claim 16, wherein the modification comprises a deletion.

18. The modified plant, plant seed, plant part, or plant cell of claim 11, wherein the plant, plant seed, plant part, or plant cell comprises a modification in at least one allele of a promoter of the TFL1b gene, wherein the modification is selected from the group consisting of:
a 30 base pair deletion from nucleotide 1539 to nucleotide 1568, as compared to reference sequence SEQ ID NO:4;
a 388 base pair deletion from nucleotide 1217 to nucleotide 1604, as compared to reference sequence SEQ ID NO:4;
a 112 base pair deletion from nucleotide 1518 to nucleotide 1629, as compared to reference sequence SEQ ID NO:4;
a 272 base pair deletion from nucleotide 951 to nucleotide 1222, as compared to reference sequence SEQ ID NO:4;
a 44 base pair deletion from nucleotide 1364 to nucleotide 1407, as compared to reference sequence SEQ ID NO:4;
a 150 base pair deletion from nucleotide 1367 to nucleotide 1516, as compared to reference sequence SEQ ID NO:4;
a 1053 base pair deletion from nucleotide 754 to nucleotide 1806, as compared to reference sequence SEQ ID NO:4;
a 104 base pair deletion from nucleotide 1216 to nucleotide 1319, as compared to reference sequence SEQ ID NO:4;
an 807 base pair deletion from nucleotide 1159 to nucleotide 1965, as compared to reference sequence SEQ ID NO:4;
a 455 base pair deletion from nucleotide 760 to nucleotide 1214, as compared to reference sequence SEQ ID NO:4;
a 90 base pair deletion from nucleotide 939 to nucleotide 1028, as compared to reference sequence SEQ ID NO:4;
a 37 base pair inversion, wherein the sequence from nucleotide 1029 to nucleotide 1065 of SEQ ID NO:4 has been deleted, inverted, and reinserted at the same location;
a 599 base pair deletion from nucleotide 1066 to nucleotide 1664, as compared to reference sequence SEQ ID NO:4;
a 601 base pair deletion from nucleotide 952 to nucleotide 1552, as compared to reference sequence SEQ ID NO:4;
a 132 base pair deletion from nucleotide 1677 to nucleotide 1808, as compared to reference sequence SEQ ID NO:4;
a 35 base pair deletion from nucleotide 1524 to nucleotide 1558, as compared to reference sequence SEQ ID NO:4;
a 930 base pair deletion from nucleotide 476 to nucleotide 1405, as compared to reference sequence SEQ ID NO:4;
a 195 base pair deletion from nucleotide 1365 to nucleotide 1559, as compared to reference sequence SEQ ID NO:4;
a 633 base pair deletion from nucleotide 928 to nucleotide 1560, as compared to reference sequence SEQ ID NO:4;
a 1221 base pair deletion from nucleotide 593 to nucleotide 1813, as compared to reference sequence SEQ ID NO:4;
a 5 base pair deletion from nucleotide 1552 to nucleotide 1556, as compared to reference sequence SEQ ID NO: 4;
a 29 base pair deletion from nucleotide 1537 to nucleotide 1565, as compared to reference sequence SEQ ID NO: 4;
a 38 base pair deletion from nucleotide 1209 to nucleotide 1246, as compared to reference sequence SEQ ID NO: 4;
a 49 base pair deletion from nucleotide 1552 to nucleotide 1600, as compared to reference sequence SEQ ID NO: 4;
a 49 base pair deletion from nucleotide 1553 to nucleotide 1601, as compared to reference sequence SEQ ID NO: 4;
an 11 base pair deletion from nucleotide 1368 to nucleotide 1378, as compared to reference sequence SEQ ID NO: 4;
a 28 base pair deletion from nucleotide 1368 to nucleotide 1395, as compared to reference sequence SEQ ID NO: 4;
a 41 base pair deletion from nucleotide 1344 to nucleotide 1384, as compared to reference sequence SEQ ID NO: 4;
a 235 base pair deletion from nucleotide 1219 to nucleotide 1453, as compared to reference sequence SEQ ID NO: 4;
a 13 base pair deletion from nucleotide 1215 to nucleotide 1227, as compared to reference sequence SEQ ID NO: 4;
a 7 base pair deletion from nucleotide 1370 to nucleotide 1376, as compared to reference sequence SEQ ID NO: 4;
a 337 base pair deletion from nucleotide 1220 to nucleotide 1556, as compared to reference sequence SEQ ID NO: 4;
a 161 base pair deletion from nucleotide 1216 to nucleotide 1376, as compared to reference sequence SEQ ID NO: 4;
a 17 base pair deletion from nucleotide 1368 to nucleotide 1384, as compared to reference sequence SEQ ID NO: 4;
a 283 base pair deletion from nucleotide 1366 to nucleotide 1648, as compared to reference sequence SEQ ID NO: 4;
a 120 base pair deletion from nucleotide 1370 to nucleotide 1489, as compared to reference sequence SEQ ID NO: 4;
a 75 base pair deletion from nucleotide 1541 to nucleotide 1615, as compared to reference sequence SEQ ID NO: 4;
a 9 base pair deletion from nucleotide 1367 to nucleotide 1375, as compared to reference sequence SEQ ID NO: 4;
a 51 base pair deletion from nucleotide 1551 to nucleotide 1601, as compared to reference sequence SEQ ID NO: 4;
a 7 base pair deletion from nucleotide 1371 to nucleotide 1377, as compared to reference sequence SEQ ID NO: 4;
a 186 base pair deletion from nucleotide 1368 to nucleotide 1553, as compared to reference sequence SEQ ID NO: 4;
a 14 base pair deletion from nucleotide 1365 to nucleotide 1378, as compared to reference sequence SEQ ID NO: 4;
a 16 base pair deletion from nucleotide 1365 to nucleotide 1380, as compared to reference sequence SEQ ID NO: 4;
a 43 base pair deletion from nucleotide 1552 to nucleotide 1594, as compared to reference sequence SEQ ID NO: 4;
a 10 base pair deletion from nucleotide 1367 to nucleotide 1376, as compared to reference sequence SEQ ID NO: 4;
a 131 base pair deletion from nucleotide 1552 to nucleotide 1682, as compared to reference sequence SEQ ID NO: 4;
a 438 base pair deletion from nucleotide 954 to nucleotide 1391, as compared to reference sequence SEQ ID NO: 4;
a 26 base pair deletion from nucleotide 1535 to nucleotide 1560, as compared to reference sequence SEQ ID NO: 4;
a 111 base pair deletion from nucleotide 1534 to nucleotide 1644, as compared to reference sequence SEQ ID NO: 4;
a 663 base pair deletion from nucleotide 937 to nucleotide 1599, as compared to reference sequence SEQ ID NO: 4;
a 98 base pair deletion from nucleotide 1551 to nucleotide 1648, as compared to reference sequence SEQ ID NO: 4;
a 34 base pair deletion from nucleotide 1526 to nucleotide 1559, as compared to reference sequence SEQ ID NO: 4;
a 79 base pair deletion from nucleotide 1528 to nucleotide 1606, as compared to reference sequence SEQ ID NO: 4;
a 61 base pair deletion from nucleotide 1542 to nucleotide 1602, as compared to reference sequence SEQ ID NO: 4;
a 381 base pair deletion from nucleotide 1214 to nucleotide 1594, as compared to reference sequence SEQ ID NO: 4;
a 187 base pair deletion from nucleotide 1368 to nucleotide 1554, as compared to reference sequence SEQ ID NO: 4;
a 109 base pair deletion from nucleotide 1369 to nucleotide 1477, as compared to reference sequence SEQ ID NO: 4;
a 5 base pair deletion from nucleotide 1550 to nucleotide 1554, as compared to reference sequence SEQ ID NO: 4;
a 1267 base pair deletion from nucleotide 734 to nucleotide 2000, as compared to reference sequence SEQ ID NO: 4;
a 190 base pair deletion from nucleotide 1371 to nucleotide 1560, as compared to reference sequence SEQ ID NO: 4;
a 36 base pair deletion from nucleotide 1537 to nucleotide 1572, as compared to reference sequence SEQ ID NO: 4;
a 46 base pair deletion from nucleotide 1541 to nucleotide 1586, as compared to reference sequence SEQ ID NO: 4;
a 5 base pair deletion from nucleotide 1552 to nucleotide 1556, as compared to reference sequence SEQ ID NO: 4;
a 955 base pair deletion from nucleotide 669 to nucleotide 1623, as compared to reference sequence SEQ ID NO: 4;
a 38 base pair deletion from nucleotide 1521 to nucleotide 1558, as compared to reference sequence SEQ ID NO: 4;
a 109 base pair deletion from nucleotide 1369 to nucleotide 1477, as compared to reference sequence SEQ ID NO: 4;
a 15 base pair deletion from nucleotide 1540 to nucleotide 1554, as compared to reference sequence SEQ ID NO: 4;
a 458 base pair deletion from nucleotide 1217 to nucleotide 1674, as compared to reference sequence SEQ ID NO: 4;
an 81 base pair deletion from nucleotide 1546 to nucleotide 1626, as compared to reference sequence SEQ ID NO: 4;
an 89 base pair deletion from nucleotide 1547 to nucleotide 1635, as compared to reference sequence SEQ ID NO: 4; and
combinations of any thereof.

19. The modified plant, plant seed, plant part, or plant cell of claim 11, wherein the plant, plant seed, plant part, or plant cell comprises a modification in the promoter of at least one allele of the TFL1b gene, wherein the modification is comprised within a genomic region between nucleotide positions 1237 and 1570 of reference sequence SEQ ID NO:4.

20. The modified plant, plant seed, plant part, or plant cell of claim 19, wherein the modification comprises a deletion of at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, or at least 150 consecutive nucleotides.

21. The modified plant, plant seed, plant part, or plant cell of claim 15, wherein the plant, plant seed, plant part, or plant cell comprises a chromosomal sequence in the TFL1b gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO:4 in the regions outside of the deletion, the insertion, the substitution, or the inversion.

22. The modified plant, plant seed, plant part, or plant cell of claim 11, wherein the plant, plant seed, plant part, or plant cell comprises a polynucleotide sequence selected from the group consisting of SEQ ID NOs:30, 31, 32, 33, 34, 35, 36, 37, 38, 39, 40, 41, 42, 43, 44, 45, 46, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, and 128.

23. (canceled)

24. The modified plant, plant seed, plant part, or plant cell of claim 15 wherein the modification reduces the expression level of the TFL1 protein, as compared to the expression level of the TFL1 protein in an otherwise identical plant, plant seed, plant part, or plant cell that lacks the modification.

25. (canceled)

26. The modified plant of claim 15, wherein the modification increases the determinacy of the plant, as compared to the determinacy of an otherwise identical plant that lacks the modification.

27-30. (canceled)

31. A polynucleotide comprising a sequence selected from the group consisting of SEQ ID NOs:30, 31, 32, 33, 34, 35, 36, 37, 38, 39, 40, 41, 42, 43, 44, 45, 46, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, and 128.

32. A guide RNA comprising a polynucleotide sequence selected from the group consisting of SEQ ID NOs:13, 14, 15, 16, 17, 18, 19, 20, 21, 22, 23, 24, 25, 26, 27, 28, and 29.

33. A method for producing a plant comprising a modified TFL1 gene, the method comprising:
a) introducing a modification into at least one target site in an endogenous TFL1 gene or a homolog thereof of a plant cell;
b) identifying and selecting one or more plant cells of step (a) comprising said modification in said TFL1 gene or homolog thereof; and
c) regenerating at least one plant from at least one or more cells selected in step (b).

34. The method of claim 33, wherein:
(a) the target site is located in a non-coding region of an endogenous TFL1 gene or homolog thereof;
(b) the modification is facilitated by the presence of at least one site-specific genome modification enzyme in said plant cell; or
(c) the modification is selected from the group consisting of a substitution, an insertion, an inversion, a deletion, a duplication, and a combination thereof.

35. The method of claim 34, wherein the non-coding region is selected from the group consisting of a promoter, an intron, a 5′-untranslated region, a 3′-untranslated region, and combinations of any thereof.

36. The method of claim 35, wherein the non-coding region is a promoter.

37. (canceled)

38. The method of claim 34, wherein the site-specific genome modification enzyme is selected from the group consisting of: an RNA-guided nuclease, a zinc-finger nuclease, a meganuclease, a TALE-nuclease, a recombinase, a transposase, and combinations of any thereof.

39. The method of claim 38, wherein the site-specific genome modification enzyme is an RNA-guided nuclease comprising a Cas nuclease, a Cpf1 nuclease, or a variant of either thereof.

40. The method of claim 34, wherein the site-specific genome modification enzyme creates at least one strand break at the target site.

41. (canceled)

42. The method of claim 34, wherein the modification is a deletion.

43. The method of claim 42, wherein the deletion comprises a region of at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, or at least 150 consecutive nucleotides.

44. A method for increasing the determinacy of a plant, comprising modifying at least one allele of an endogenous promoter of a TFL1 gene, such that TFL1 protein production is reduced as compared to the production of TFL1 protein production in an otherwise identical plant that lacks the modification.
